(19) United States
(12) Patent Application Publication
(10) Pub. No.: US 2025/0244236 A1
(43) Pub. Date: Jul. 31, 2025

(54) SYSTEMS, DEVICES AND METHODS FOR ANALYZING URINE

(71) Applicant: URAY TECHNOLOGIES PTE. LTD., Singapore (SG)

(72) Inventors: Igor Sergeyevich BALASHOV, Balashikha (RU); Iurii Mikhailovich POIMANOV, Yekaterinburg (RU); Andrei Anatolevich GRUNIN, Moscow (RU)

(21) Appl. No.: 19/039,565

(22) Filed: Jan. 28, 2025

Related U.S. Application Data
(63) Continuation-in-part of application No. PCT/IB2023/057656, filed on Jul. 27, 2023.
(60) Provisional application No. 63/393,098, filed on Jul. 28, 2022.

(51) Int. Cl. G01N 21/3577; G01N 21/31; G01N 21/359; G01N 33/493; G16H 10/60

(52) U.S. Cl. CPC G01N 21/3577; G01N 21/359; G01N 33/493; G16H 10/60; G01N 2021/3155; G01N 2201/1296

(57) ABSTRACT

Systems, methods and devices for analyzing urine by generating a urine data matrix based on personal patient data and on a urine spectrum received by a light detector from a light source or urine spectra received by a light detector from light sources. The generated urine data matrix is fed to one or more computing models to determine at least one patient urine parameter or detect at least one patient disease.

### CROSS-REFERENCE TO RELATED APPLICATIONS

This application is a continuation-in-part of International (PCT) application No. PCT/IB2023/057656 filed on Jul. 27, 2023, which claims priority to U.S. provisional patent application No. 63/393,098 filed on Jul. 28, 2022. Each of these applications are incorporated herein by reference for all purposes.

### FIELD OF THE INVENTION

The present invention generally relates to computer-aided urine analysis, and more particularly to systems, devices and methods for analyzing urine by using computing models.

### BACKGROUND OF THE INVENTION

Urine is a liquid by-product of metabolism in humans and other mammals. Urine flows from kidneys through a ureter to a urinary bladder. Urination results in urine being excreted from a mammalian body through a urethra.

A urine sample may be collected by a patient at home or office of a healthcare provider. Healthcare provider typically give out containers for urine samples to be collected therein. The urine sample collected in the container usually sent to a special laboratory for a urine analysis by using at least one of known urine analysis techniques.

Generally, the urine analysis is a test that examines visual, chemical and microscopic aspects of urine of a patient. The urine analysis includes a variety of tests that detect and measure various urine parameters by using a urine sample.

Because the urine analysis is easy, cheap, and productive, it is usually recommended by healthcare providers as a part of an initial examination for all patients and may be repeated as clinically warranted. A result of the urine analysis may require more sophisticated chemical, immunologic, or bacteriologic studies to be performed for the patient.

Healthcare providers commonly use the urine analysis to diagnose urinary tract infections and to screen for or monitor certain common health conditions, such as a liver disease, kidney disease and diabetes. In particular, the urine analysis may be required for the following reasons:
checking an overall health of the patient. The urine analysis may be a part of a routine medical exam, pregnancy checkup or pre-surgery preparation. Furthermore, when the patient is admitted to a hospital, the urine analysis may be used to screen for a variety of disorders, such as diabetes, kidney disease or liver disease;
diagnosing a medical condition of the patient. The urine analysis may be requested if the patient has an abdominal pain, back pain, frequent or painful urination, blood in urine, or other urinary problems. In other words, the urine analysis can help diagnose the cause of these signs and symptoms.
monitoring a medical condition. If the patient is diagnosed with a medical condition, such as kidney disease or a urinary tract infection, a healthcare provider may recommend testing patient's urine regularly, thereby allowing a condition and treatment of the patient to be monitored.

The urine sample may be examined by a lab technician for various properties, solutes, cells, casts, crystals, organisms, or particulate matter. For example, the urine sample may be examined for the following urine parameters:
Acidity (pH). The pH level indicates the amount of acid in urine. The pH level may indicate a kidney or urinary tract disorder.
Specific gravity (concentration). A measure of concentration shows how concentrated the particles are in urine of the patient. A higher than normal concentration often is a result of not drinking enough fluids.
Protein. Low levels of protein in urine are typical for the most patients. Small increases in protein in urine usually are not a cause for concern, but larger amounts may indicate a kidney problem.
Glucose. The amount of glucose (sugar) in urine of the patient is typically too low to be detected. Any detection of sugar on this test usually calls for follow-up testing for diabetes.
Ketones. As with sugar, any amount of ketones detected in urine of the patient may be a sign of diabetes and requires follow-up testing.
Bilirubin. Bilirubin is a product of red blood cell breakdown. Usually, bilirubin is carried in the blood and passes into liver where it is removed and becomes part of bile. Bilirubin in urine of the patient may indicate liver damage or disease.
Nitrites or leukocyte esterase. Either nitrites or leukocyte esterase—a product of white blood cells—in urine of the patient may indicate a urinary tract infection.
Blood. Blood in urine of the patient requires additional testing. It may be a sign of kidney damage, infection, kidney or bladder stones, kidney or bladder cancer, or blood disorders.
White blood cells (leukocytes). Leukocytes in urine of the patient may be a sign of an infection.
Red blood cells (erythrocytes). Erythrocytes in urine of the patient may be a sign of kidney disease, a blood disorder or another underlying medical condition, such as bladder cancer.
Bacteria, yeast or parasites. Bacteria, yeast and/or parasites in urine of the patient may indicate an infection.
Casts—tube-shaped proteins. Casts in urine of the patient may be a result of kidney disorders.
Crystals. Crystals that form from chemicals in urine of the patient may be a sign of kidney stones.

It is to note that the urine analysis alone usually does not provide a definite diagnosis. Depending on signs and symptoms of the patient, a healthcare provider may recommend examination of the urine sample for a particular combination of urine parameters. However, evaluation of urine parameters recommended by the healthcare provider does not guarantee that a medical condition or disease of the patient will be precisely diagnosed. Furthermore, there may be cases where the patient may not have any noticeable symptoms or may need to examine different urine parameters regularly in order to monitor the development of patient's disease, wherein the patient may not have time or money resources enough for visiting the healthcare provider. In such cases, non-invasive devices based on computer-aided urine analysis methods may be effectively used by the patient for examining the urine sample of the patient for a plurality of urine parameters at once, thereby replacing an initial reference to a healthcare provider and, thus, an initial medical disposal for examination of particular urine parameters.

Meanwhile, most of the known devices, methods and systems for analyzing urine samples still have a problem with certain or correct urine parameter determinations.

The problem of the known devices, methods and systems is generally conditioned by the fact that they have at least a complicated optical system to be used for conducting a light and the fact that they use a processing or computer device having an unsuitable or complicated configuration.

Consequently, a main technical problem to be solved by the present invention is to develop devices, methods and systems for analyzing urine that would at least partly eliminate the above disadvantage of the prior art, i.e. eliminate uncertain or incorrect urine parameter determinations.

A further technical problem underlying the present invention is seeking an alternative to the devices, methods and systems for analyzing urine as disclosed in the art.

### SUMMARY OF THE INVENTION

It is an object of the present invention to develop devices, methods and systems for analyzing urine that would solve at least one of the above technical problems, in particular the main technical problem.

To achieve the objective of the invention, as embodied and broadly described herein, in a first aspect of the present invention, there is provided a device for analyzing urine, the device comprising: (1) a base for receiving a urine container; (2) a first light source designed to emit a NIR broadband light in a direction of the urine container; (3) a second light source designed to emit a VIS broadband light in a direction of the urine container; (4) a light detector configured to detect the NIR light passed through urine in the urine container to generate a first urine spectrum and the VIS light passed through urine in the urine container to generate a second urine spectrum, wherein the first light source, the second light source and the light detector are mounted on the base such that the received container is positioned between the light sources and the light detector, and wherein the device further comprises; and (5) a processing device connected to the light detector to receive the first and second urine spectra therefrom, configured to receive personal patient data corresponding to the received urine spectra and configured to combine the received urine spectra with the received personal patient data to generate a urine data matrix, wherein the processing device is further configured to feed the urine data matrix to one or more computing models stored in a data storage accessed by the processing device, each computing model being based on predetermined personal patient data and a predetermined correlation between a particular urine parameter obtained by a standard urine analysis method and measured urine spectra corresponding to the personal patient data, so as to determine at least one urine parameter.

In an embodiment of the present invention according to the first aspect, the first and second light sources in the device according to the first aspect of the present invention are both mounted on the same printed circuit board.

In another embodiment of the present invention according to the first aspect, each of the first and second urine spectra in the device according to the first aspect of the present invention comprises spectral data related to a transmission spectrum, scattering spectrum, fluorescence spectrum and/or a luminescence spectrum.

In one embodiment of the present invention according to the first aspect, the first light source in the device according to the first aspect of the present invention is a first combination of narrowband light sources, each emitting light in a predetermined part of a NIR wavelength region, and the second light source is a second combination of narrowband light sources, each emitting light in a predetermined part of the VIS light wavelength region.

In still another embodiment of the present invention according to the first aspect, the light detector in the device according to the first aspect of the present invention is single or multiple broadband light detectors designed to detect light in a NIR light wavelength region and detect light in a VIS light wavelength region, wherein the first light source is a first combination of narrowband light sources, each emitting light in a predetermined part of a NIR wavelength region, and the second light source is a second combination of narrowband light sources, each emitting light in a predetermined part of the VIS light wavelength region.

In yet another embodiment of the present invention according to the first aspect, the light detector in the device according to the first aspect of the present invention is a combination of narrowband light detectors, each being designed to detect light in a predetermined part of the NIR light wavelength region or detect light in a predetermined part of VIS light wavelength region.

In various embodiments of the present invention according to the first aspect, the personal patient data in the device according to the first aspect of the present invention comprises height, weight, gender, age and/or diagnosis.

In some embodiments of the present invention according to the first aspect, the device further comprises a diffuser positioned between the urine container and the light detector, the diffuser being designed to scatter or homogenize the light passed through urine in the urine container.

In some other embodiments of the present invention according to the first aspect, the processing device is further configured to preliminary normalize each of the received first and second urine spectra by maximum intensity before generating the urine data matrix.

In other embodiments of the present invention according to the first aspect, the processing device is configured to apply each of the computing models to the urine data matrix by categorizing the urine matrix according to pre-determined target urine parameters into a category of molecular-scale biomarkers and a category of cell-size biomarkers and by feeding the urine matrix to two PLS regression models, each corresponding to one of the determined urine parameter category and being based on a predetermined correlation between particular urine parameters obtained by standard urine analysis methods and measured urine spectra, for determining said at least one urine parameter.

In any other embodiments of the present invention according to the first aspect, each of the computing models used by the processing device for determining said at least one urine parameter is preliminary trained by using a training dataset formed of urine sample spectra obtained by a standard spectrometer and synthetic spectra, each synthetic spectrum being formed by combining at least two of the obtained urine sample spectra related to the same urine parameter category determined by the processing device.

In a second aspect of the present invention, there is provided a device for analyzing urine, the device comprising: (1) a base for receiving a urine container; (2) a light source designed to emit a UV-VIS-NIR light in a direction of the urine container; (3) a light detector configured to detect the UV-VIS-NIR light passed through urine in the urine container to generate a urine spectrum, wherein the light source and the light detector are mounted on the base such that the received container is positioned between the light source and the light detector; and (4) a processing device connected to the light detector to receive the urine spectrum therefrom, configured to receive personal patient data corresponding to the received urine spectrum and configured to combine the received urine spectrum with the received personal patient data to generate a urine data matrix, wherein the processing device is further configured to feed the urine data matrix to one ore more computing models stored in a data storage accessed by the processing device, each computing model being based on predetermined personal patient data and a predetermined correlation between a particular urine parameter obtained by a standard urine analysis method and measured urine spectra corresponding to the personal patient data, so as to determine at least one urine parameter.

In an embodiment of the present invention according to the second aspect, the light source in the device according to the second aspect of the present invention is a single light source or a combination of light sources.

In another embodiment of the present invention according to the second aspect, the generated urine spectrum in the device according to the second aspect of the present invention comprises spectral data related to a transmission spectrum, scattering spectrum, fluorescence spectrum and/or a luminescence spectrum.

In one embodiment of the present invention according to the second aspect, the light source in the device according to the second aspect of the present invention is a combination of narrowband light sources, each emitting light in a predetermined part of the UV-VIS-NIR light wavelength region.

In still another embodiment of the present invention according to the second aspect, the light detector in the device according to the second aspect of the present invention is a broadband light detector designed to detect light in a UV-VIS-NIR light wavelength region, wherein the light source is a combination of narrowband light sources, each emitting light in a predetermined part of the UV-VIS-NIR light wavelength region.

In yet another embodiment of the present invention according to the second aspect, the light detector in the device according to the second aspect of the present invention is a combination of narrowband light detectors, each being designed to detect light in a predetermined part of a UV-VIS-NIR light wavelength region.

In various embodiments of the present invention according to the second aspect, the personal patient data in the device according to the second aspect of the present invention comprises height, weight, gender, age and/or diagnosis.

In other embodiments of the present invention according to the second aspect, the device further comprises a diffuser positioned between the urine container and the light detector, the diffuser being designed to scatter or homogenize the light passed through urine in the urine container.

In a third aspect of the present invention, there is provided a method of analyzing a urine, the method comprising: (i) emitting, by means of a first light source, a NIR broadband light in a direction of a urine container; (ii) emitting, by means of a second light source, a VIS broadband light in a direction of the urine container; (iii) detecting, by means of a light detector, the NIR broadband light passed through urine in the urine container to generate a first urine spectrum and the VIS light passed through urine in the urine container to generate a second urine spectrum, wherein the first light source, the second light source and the light detector are mounted on the base such that the received container is positioned between the light sources and the light detector; (iv) transmitting the first and second urine spectra from the light detector to a computer device connected to the light detector; (v) receiving, by the computer device, personal patient data corresponding to the received urine spectra from a data storage accessed by the computer device (vi) combining, by the computer device, the transmitted urine spectra with the received personal patient data to generate a urine data matrix; and (vii) feeding, by the computer device, the urine data matrix to one or more computing models stored in a data storage accessed by the computer device, each computing model being based on predetermined personal patient data and a predetermined correlation between a particular urine parameter obtained by a standard urine analysis method and measured urine spectra corresponding to the personal patient data and, so as to determine at least one urine parameter.

In a fourth aspect of the present invention, there is provided a method of analyzing a urine, the method comprising: (i) emitting, by means of a light source, a UV-VIS-NIR light in a direction of a urine container; (ii) detecting, by means of a light detector, the UV-VIS-NIR light passed through urine in the urine container to generate a urine spectrum; (iii) transmitting the urine spectrum from the light detector to a computer device connected to the light detector, wherein the light source and the light detector are mounted on the base such that the received container is positioned between the light source and the light detector; (iv) receiving, by the computer device, personal patient data corresponding to the received urine spectrum from a data storage accessed by the computer device (v) combining, by the computer device, the transmitted urine spectrum with the received personal patient data to generate a urine data matrix; and (vi) feeding, by the computer device, the urine data matrix to one or more computing models stored in a data storage accessed by the computer device, each computing model being based on predetermined personal patient data and a predetermined correlation between a particular urine parameter obtained by a standard urine analysis method and measured urine spectra corresponding to the personal patient data and, so as to determine at least one urine parameter.

In a fifth aspect of the present invention, there is provided a system for analyzing urine, the system comprising: (1) a first light source designed to emit a NIR broadband light in a direction of a urine container; (2) a second light source designed to emit a VIS broadband light in a direction of the urine container; (3) a light detector designed to detect the NIR light passed through urine in the urine container to generate a first urine spectrum and the VIS light passed through urine in the urine container to generate a second urine spectrum, wherein the first light source, the second light source and the light detector are mounted such that the received container is positioned between the light sources and the light detector; and (4) a computer device connected to the light detector to receive the first and second urine spectra therefrom, configured to receive personal patient data corresponding to the received urine spectra from a data storage accessed by the computer device and configured to combine the received urine spectra with the received personal patient data to generate a urine data matrix, wherein the computer device is further configured to feed the generated urine data matrix to one or more computing models stored in a data storage accessed by the computer device, each computing model being based on predetermined personal patient data and a predetermined correlation between a particular urine parameter obtained by a standard urine analysis method and measured urine spectra corresponding to the personal patient data and, so as to determine at least one urine parameter.

In a sixth aspect of the present invention, there is provided a system for analyzing urine, the system comprising: (1) a light source designed to emit a UV-VIS-NIR light in a direction of a urine container; (2) a light detector designed to detect the UV-VIS-NIR light passed through urine in the urine container to generate a urine spectrum, wherein the light source and the light detector are mounted such that the received container is positioned between the light source and the light detector; and (3) a computer device connected to the light detector to receive the generated urine spectrum therefrom, configured to receive personal patient data corresponding to the received urine spectrum and configured to combine the received urine spectrum with the received personal patient data to generate a urine data matrix, wherein the computer device is further configured to feed the generated urine data matrix to one or more computing models stored in a data storage accessed by the computer device, each computing model being based on predetermined personal patient data and a predetermined correlation between a particular urine parameter obtained by a standard urine analysis method and measured urine spectra corresponding to the personal patient data, so as to determine at least one urine parameter.

In a seventh aspect of the present invention, there is provided a device for analyzing urine, the device comprising: (1) a base for receiving a urine container; (2) a first light source designed to emit a NIR broadband light in a direction of the urine container; (3) a second light source designed to emit a VIS broadband light in a direction of the urine container; (4) a light detector configured to detect the NIR light passed through urine in the urine container to generate a first urine spectrum and the VIS light passed through urine in the urine container to generate a second urine spectrum, wherein the first light source, the second light source and the light detector are mounted on the base such that the received container is positioned between the light sources and the light detector; and (5) a processing device connected to the light detector to receive the first and second urine spectra therefrom, configured to receive personal patient data corresponding to the received urine spectra and configured to combine the received urine spectra with the received personal patient data to generate a urine data matrix, wherein the processing device is further configured to feed the urine data matrix to one or more computing models stored in a data storage accessed by the processing device, each computing model being based on predetermined personal patient data and a predetermined correlation between a particular disease and measured urine spectra corresponding to the personal patient data, so as to detect at least one disease.

In other embodiments of the present invention according to the seventh aspect, the device further comprises a diffuser positioned between the urine container and the light detector, the diffuser being designed to scatter or homogenize the light passed through urine in the urine container.

In an eight aspect of the present invention, there is provided a device for analyzing urine, the device comprising: (1) a base for receiving a urine container; (2) a light source designed to emit a UV-VIS-NIR light in a direction of the urine container; (3) a light detector configured to detect the UV-VIS-NIR light passed through urine in the urine container to generate a urine spectrum, wherein the light source and the light detector are mounted on the base such that the received container is positioned between the light source and the light detector; and (4) a processing device connected to the light detector to receive the urine spectrum therefrom, configured to receive personal patient data corresponding to the received urine spectrum and configured to combine the received urine spectrum with the received personal patient data to generate a urine data matrix, wherein the processing device is further configured to feed the urine data matrix to one or more computing models stored in a data storage accessed by the computer device, each computing model being based on predetermined personal patient data and a predetermined correlation between a particular disease and measured urine spectra corresponding to the personal patient data, so as to detect at least one disease.

In other embodiments of the present invention according to the eight aspect, the device further comprises a diffuser positioned between the urine container and the light detector, the diffuser being designed to scatter or homogenize the light passed through urine in the urine container.

In a ninth aspect of the present invention, there is provided a method of analyzing a urine, the method comprising: (i) emitting, by means of a first light source, a NIR broadband light in a direction of a urine container; (ii) emitting, by means of a second light source, a VIS broadband light in a direction of the urine container; (iii) detecting, by means of a light detector, the NIR broadband light passed through urine in the urine container to generate a first urine spectrum and the VIS light passed through urine in the urine container to generate a second urine spectrum, wherein the first light source, the second light source and the light detector are mounted on the base such that the received container is positioned between the light sources and the light detector; (iv) transmitting the first and second urine spectra from the light detector to a computer device connected to the light detector; (v) receiving, by the computer device, personal patient data corresponding to the received urine spectra; (vi) combining, by the computer device, the transmitted urine spectra with the received personal patient data to generate a urine data matrix; and (vii) feeding, by the computer device, the urine data matrix to one or more computing models stored in a data storage accessed by the computer device, each computing model being based on predetermined personal patient data and a predetermined correlation between a particular disease and measured urine spectra corresponding to the personal patient data corresponding to the personal patient data, so as to detect at least one disease.

In a tenth aspect of the present invention, there is provided a method of analyzing a urine, the method comprising: (i) emitting, by means of a light source, a UV-VIS-NIR light in a direction of a urine container; (ii) detecting, by means of a light detector, the UV-VIS-NIR light passed through urine in the urine container to generate a urine spectrum; (iii) transmitting the urine spectrum from the light detector to a computer device connected to the light detector, wherein the light source and the light detector are mounted on the base such that the received container is positioned between the light source and the light detector; (iv) receiving, by the computer device, personal patient data corresponding to the received urine spectrum; (v) combining, by the computer device, the transmitted urine spectrum with the received personal patient data to generate a urine data matrix; and (vi) feeding, by the computer device, the urine data matrix to one or more computing models stored in a data storage accessed by the computer device, each computing model being based on predetermined personal patient data and a predetermined correlation between a particular disease and measured urine spectra corresponding to the personal patient data, so as to detect at least one disease.

In a eleventh aspect of the present invention, there is provided a system for analyzing urine, the system comprising: (1) a first light source designed to emit a NIR broadband light in a direction of a urine container; (2) a second light source designed to emit a VIS broadband light in a direction of the urine container; (3) a light detector designed to detect the NIR light passed through urine in the urine container to generate a first urine spectrum and the VIS light passed through urine in the urine container to generate a second urine spectrum, wherein the first light source, the second light source and the light detector are mounted such that the received container is positioned between the light sources and the light detector; and (4) a computer device connected to the light detector to receive the first and second urine spectra therefrom, configured to receive personal patient data corresponding to the received urine spectra and configured to combine the received urine spectra with the received personal patient data to generate a urine data matrix, wherein the computer device is further configured to feed the generated urine data matrix to one or more computing models stored in a data storage accessed by the computer device, each computing model being based on predetermined personal patient data and a predetermined correlation between a particular disease and measured urine spectra corresponding to the personal patient data, so as to detect at least one disease.

In a twelfth aspect of the present invention, there is provided a system for analyzing urine, the system comprising: (1) a light source designed to emit a UV-VIS-NIR light in a direction of a urine container; (2) a light detector designed to detect the UV-VIS-NIR light passed through urine in the urine container to generate a urine spectrum, wherein the light source and the light detector are mounted such that the received container is positioned between the light source and the light detector; and (3) a computer device connected to the light detector to receive the generated urine spectrum therefrom, configured to receive personal patient data corresponding to the received urine spectrum and configured to combine the received urine spectrum with the received personal patient data to generate a urine data matrix, wherein the computer device is further configured to feed the generated urine data matrix to one or more computing models stored in a data storage accessed by the computer device, each computing model being based on predetermined personal patient data and a predetermined correlation between a particular disease and measured urine spectra corresponding to the personal patient data, so as to detect at least one disease.

The present invention according to any of the above-disclosed first-sixth aspects provides a main technical effect which is improved reliability due to reduced false determinations of urine parameters to be determined by the device for analyzing urine due to the following positive technical aspects: (a) use of a simplified and optimized computing model using a urine data matrix to be fed thereto as an input; and (b) use of the simplified optical system where the light source(s), the light detector and the urine container are mounted on the same base such that the urine container is positioned between the light source(s) and the light detector, and the light emitted by the light source(s) is directed to the urine container. The present invention according to any of the above-disclosed seventh-twelfth aspects also provides the above main technical effect due to the following positive technical aspects: (a) use of a simplified and optimized computing model using a urine data matrix to be fed thereto as an input; and (b) use of the simplified optical system where the light source(s), the light detector and the urine container are mounted on the same base such that the urine container is positioned between the light source(s) and the light detector, and the light emitted by the light source(s) is directed to the urine container.

Furthermore, a further technical effect provided by the present invention according to any of the first-twelfth aspects is an improved accuracy of urine parameter determination.

### BRIEF DESCRIPTION OF THE DRAWINGS

While the specification concludes with claims particularly pointing out and distinctly claiming the present invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the drawings, wherein:

FIG. 1 shows a block diagram of a urine-analyzing system according to a first aspect of the present invention;

FIG. 2 shows a block diagram of a urine-analyzing device according to a second aspect of the present invention;

FIG. 3A is graph of intensity vs. wavelength illustrating ten urine spectra of the same urine sample as generated by a light detector in case when a diffuser is not installed between a urine container and the light detector;

FIG. 3B is graph of intensity vs. wavelength illustrating ten urine spectra of the same urine sample as generated by a light detector in case when a diffuser is installed between a urine container and the light detector;

FIG. 3C is graph of intensity vs. wavelength illustrating urine spectra for different volumes of urine sample, the urine spectra being generated in case when a diffuser is not installed between a urine container and the light detector;

FIG. 3D is another graph of intensity vs. wavelength illustrating normalized urine spectra for different volumes of urine sample, the urine spectra being generated in case when a diffuser is not installed between a urine container and the light detector;

FIG. 3E is graph of intensity vs. wavelength illustrating urine spectra for different volumes of urine sample, the urine spectra being generated in case when a diffuser is installed between a urine container and the light detector;

FIG. 3F is another graph of intensity vs. wavelength illustrating normalized urine spectra for different volumes of urine sample, the urine spectra being generated in case when a diffuser is installed between a urine container and the light detector;

FIG. 3G is a data processing scheme by using a preliminary categorization and a PLS model;

FIG. 4 is a flow diagram of a method of analyzing a urine according to a fifth aspect of the present invention;

FIG. 5 is a flow diagram of a method of analyzing a urine according to a sixth aspect of the present invention;

FIG. 6 shows a block diagram of a urine-analyzing system according to a fifth aspect of the present invention;

FIG. 7 shows a block diagram of a urine-analyzing device according to a sixth aspect of the present invention;

FIG. 8 is a flow diagram of a method of analyzing a urine according to a eleventh aspect of the present invention; and

FIG. 9 is a flow diagram of a method of analyzing a urine according to a twelfth aspect of the present invention.

### DETAILED DESCRIPTION OF THE PREFERRED EMBODIMENTS

The present invention will now be described more fully with reference to the accompanying drawings, in which example embodiments of the present invention are illustrated. The subject matter of this disclosure may, however, be embodied in many different forms and should not be construed as being limited to the embodiments set forth herein.

The following example embodiments of the present invention are provided for analyzing urine by using a urine sample collected by a patient at home or office of a healthcare provider. For example, the patient may collect the urine sample by using a urine container provided by the healthcare provider or purchased by the patient in a drugstore. Thus, the urine sample contained in the urine container is analyzed by the present invention, thereby allowing at least one parameter of the patient urine (i.e. a urine biomarker) to the identified, at least one disease of the patient to be identified, or values of at least one parameter of the patient urine to be determined.

In the context of this document, unless explicitly stated otherwise, the term “patient” means first of all a potentially sick person (a member of the mammalian class) seeking medical advice or remaining under medical observation to have a disease diagnosed and/or treated, wherein the term “patient” also means secondly potentially sick mammalian animals remaining under medical observation to diagnose and/or treat their disease. Meanwhile, in context of this document, unless stated otherwise, the term “patient” also means thirdly any human users (e.g. pregnant woman, disabled person, chronic person, healthy person, sportsman, and etc.) wishing to learn their health status.

Furthermore, in the context of this document, unless expressly stated otherwise, the term “mammal” means a human or an animal, in particular anthropoid and non-human primates, dogs, cats, horses, camels, donkeys, cows, sheep, pigs, and other well-known mammals.

In the context of this document, unless explicitly stated otherwise, the term “urine parameter” means any one of the following urine parameters determined during a standard or special analysis of the urine: glucose (sugar), leukocyte esterase, bilirubin, urobilirubin, blood, protein, red blood cells (erythrocytes), white blood cells (leukocytes), squamous epithelial cells, casts, crystals, bacteria, yeast, parasites, nitrates, nitrite, ketones, specific gravity (density), acidity (pH), concentration, mucus, pathological cylinders, small cells, squamous cells, hyaline casts, microalbumin, creatinine, estimated glomerular filtration rate (eGFR) and other appropriate urine factors or parameters known in the art.

Furthermore, in the context of this document, unless expressly stated otherwise, the term “UV-VIS-NIR light” means Ultraviolet/Visible/Near-Infrared light having a wavelength in the range from 200 nm to 1000 nm; the term “NIR broadband light” means near-infrared broadband light having a wavelength in the range from 670 nm to 1000 nm; the term “VIS broadband light” means visible broadband light having a wavelength in the range from 400 nm to 700 nm.

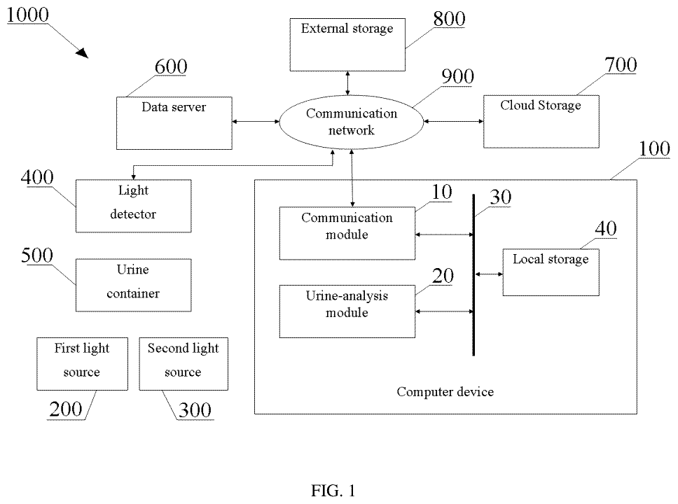

FIG. 1 is a block diagram illustrating a system 1000 for analyzing urine according to a first aspect of the present invention, the urine-analyzing system comprising the following functional devices: a computer device 100, a first light source 200, a second light source 300, a light detector 400, a urine container 500, a data server 600, a cloud storage 700, and an external storage 800.

In the system 1000 according to the first aspect of the present invention, the computer device 100 is configured to process urine data corresponding to a urine sample or urine of the patient in the below-described manner, thereby allowing (i) detection or identification of at least one urine parameter related to the patient urine, (ii) determination of values of at least one urine parameter related to the patient urine (i.e. evaluation of patient urine parameters) or (iii) detection of at least one disease related to the patient. In other words, depending on a particular alternative implementation of the computer device 100 shown in FIG. 1, the computer device 100 uses urine data corresponding to the patient to allow at least one urine parameter to be identified, at least one urine parameter to be evaluated or at least one patient disease to be detected or diagnosed.

The urine container 500 shown in FIG. 1 is installed or mounted between the light detector 400 and the light sources 200, 300 such that light emitted by any of the first and second light sources 200, 300 is allowed to pass through urine contained in the urine container 500. Before the urine container 500 is illuminated by light emitted by any of the light sources 200, 300, the urine container 500 is required to be filled by the urine or urine sample to be analyzed. For example, the urine container 500 may be in the form of a transparent urine container having a cylindrical surface and a standard diameter of 50 mm, wherein the urine container 500 may be made of plastic.

It is to note that the positioning of the urine container 500 between the light detector 400 and the light sources 200, 300 allows the light detector 400 to generate raw or original urine spectra and, therefore, allows the urine-analysis module 20 to generate the urine data matrix based on the generated raw or original urine spectra (i.e. without applying any pre-processing or filtering procedures to the generated urine spectra).

The first light source 200 shown in FIG. 1 is designed to emit a NIR (near-infrared) broadband light in a direction of the urine container 500, wherein the emitted NIR broadband light has a wavelength in the range of 670-1000 nm.

The second light source 300 shown in FIG. 1 is designed to emit a VIS (visible) broadband light in a direction of the urine container 500, wherein the emitted VIS broadband light has a wavelength in the range of 400-700 nm.

In other words, use of a combination of the first and second light sources 200, 300 shown in FIG. 1 allows covering the wavelength range from 400 nm to 1000 nm.

Moreover, use the first NIR light source 200 and the second VIS light source 300 separately mounted on the base 150 reduces a volume of spectral data contained in the urine spectrum generated by the light detector 400 as compared to a single VIS-NIR light source, thereby reducing a number of false determinations of a urine parameter that would be conditioned by extra spectral data contained in the urine spectrum to be used for generating the urine data matrix.

For example, the first and second light sources 200, 300 shown in FIG. 1 may be each in the form of a LED light source (i.e. light source in the form of light-emitting diode) designed to emit light in a corresponding wavelength range being specific for some urine parameters. In another example, the first and second light sources 200, 300 may be each in the form of an incandescent lamp or laser designed to emit light in a corresponding wavelength range.

In one embodiment of the present invention, the first and second light sources 200, 300 may be both installed or mounted on the same printed circuit board. In another embodiment of the present invention, the first and second light sources 200, 300 may be each installed or mounted on a separate circuit board.

In the system 1000 according to the first aspect of the present invention, the light detector 400 is designed to detect the NIR light passed through urine in the urine container 500 to generate a first urine spectrum and detect the VIS light passed through urine in the urine container 500 to generate a second urine spectrum. It is to note that the NIR light and the VIS light may be emitted simultaneously or alternatively (in series) by the first light source 200 and the second light source 300, respectively.

For example, the light detector 400 shown in FIG. 1 may be in the form of a multichannel spectrometer designed to detect light in a wavelength range of 400-1000 nm, i.e. light in the wavelength range being specific for the VIS broadband light emitted by the second light source 300 and light in the wavelength range being specific for the NIR broadband light emitted by the first light source 200. In another example, the light detector 400 may be in the form of multispectral sensor designed to receive light in a wavelength range of 400-1000 nm.

In a preferred example, the light detector 400 shown in FIG. 1 may be in the form of an 18-channel spectrometer. Such 18-channel spectrometer may comprise three (3) measuring units mounted on the same board, each having six (6) independent optical filters with FWHM of 20 nm for each channel, so that these 18 optical channels allow a urine spectrum to be measured in the range from 400 nm to 1000 nm.

In other examples, the light detector 400 shown in FIG. 1 may be in the form of a low-resolution light detector.

In one embodiment of the present invention, the light detector 400 may be a combination of discrete/narrowband light detectors, each being designed to detect light in a predetermined part of the NIR light wavelength region or detect light in a predetermined part of the VIS light wavelength region.

It is to note that each of the first and second urine spectra generated by the light detector 400 shown in FIG. 1 comprises spectral data related to transmission spectrum, scattering spectrum, fluorescence spectrum and/or a luminescence spectrum. In a preferred embodiment of the present invention, each of the first and second urine spectra generated by the light detector 400 shown in FIG. 1 is a transmission spectrum or scattering spectrum. In other embodiments of the present invention, each of the first and second urine spectra generated by the light detector 400 is a transmission optical spectrum, i.e. the light detector 400 is designed to detect intensity of light transmitted or passed through urine contained in the urine container 500.

In an embodiment of the present invention, the first light source 200 may be a first combination of discrete/narrowband light sources, each emitting light in a predetermined part of the NIR wavelength region (e.g. an operating bandwidth may be less than 50 nm), and the second light source 300 is a second combination of discrete/narrowband light sources, each emitting light in a predetermined part of the VIS light wavelength region (e.g. an operating bandwidth may be less than 50 nm). Furthermore, in the present embodiment of the present invention, the light detector 400 shown in FIG. 1 may be a single broadband light detector or multiple broadband light detectors designed to detect light in the NIR light wavelength region and detect light in the VIS light wavelength region. As an alternative, in the present embodiment of the present invention, the light detector 400 shown in FIG. 1 may be a combination of discrete/narrowband light detectors, each being designed to detect light in a predetermined part of the NIR light wavelength region or detect light in a predetermined part of the VIS light wavelength region.

As shown in FIG. 1, the light detector 400 is communicatively connected, via a communication network 900, to the computer device 100, so that urine spectra generated by the light detector 400 are transmitted via the communication network 900 to the computer device 100. In one embodiment of the present invention according to the first aspect, the light detector 400 may be connected to the computer device 100 in wire manner. The computer device 100 shown in FIG. 1 receives the first and second urine spectra generated by the light detector 400 and is configured to process the received urine spectra in the below-described manner.

It is to note that the operation of the light sources 200, 300 and the operation of the light detector 400 may be controlled remotely, for example by using the computer device 100 or at least one external control device communicatively connected to said functional components in a wire or wire-less manner and configured to control at least one of said functional components. Alternatively, the operation of the light sources 200, 300 may be each individually controlled by using a special control device mounted together with a corresponding light source controlled by the special control device on the same circuit board or mounted inside a housing of a corresponding light source controlled by the special control device; the operation of the light detector 400 may be controlled by using a separate control unit mounted inside a housing of the light detector 400 and connected to the light detector 400 in wire manner. Alternatively, the operation of the light sources 200, 300 may be controlled by using a special control device mounted together with both light sources controlled by the special control device on the same circuit board.

In the system 1000 according to the first aspect of the present invention, the computer device 100 is comprised of two main functional modules: a communication module 10 and a urine-analysis module 20. The computer device 100 also comprises a local storage 40 and a communication bus 30, wherein the urine-analysis module 20 is communicatively coupled to the communication module 10 via the communication bus 30, and the communication module 10 and the urine-analysis module 20 are each communicatively coupled to the local storage 40 via the communication bus 30.

Functionalities of the communication module 10 and urine-analysis module 20 will be fully described below with reference to FIG. 1.

The communication module 10 shown in FIG. 1 may be communicatively connected, via the communication network 900, to the data server 600, the cloud storage 700, the external storage 800 or other similar external devices used for storing urine spectra generated by the light detector 400 to receive therefrom at least two urine spectra (namely, at least the above first and second urine spectra) to be processed by the computer device 100. In one embodiment of the present invention according to the first aspect, the communication module 10 may be connected directly to the data server 600, the cloud storage 700 or the external storage 800 in a wire manner.

The communication network 900 shown in FIG. 1 may be in the form of Internet, 3G network, 4G network, 5G network, Wi-Fi network, Bluetooth network or any other wire or wireless network supporting appropriate data communication technologies or protocols.

The communication module 10 shown in FIG. 1 may be implemented as a network adapter provided with slots appropriate for connecting physical cables of desired types thereto if wired connections are provided between the computer device 100 and any external devices mentioned in the present document. Alternatively, if wireless connections are provided between the computer device 100 and any external devices mentioned in the present document, the communication module 10 may be implemented as a network adapter in form of Wi-Fi-adaptor, 3G/4G/5G-adaptor, LTE-adaptor, Bluetooth adaptor or any another appropriate adaptor supporting any known wireless communication technology or protocol. In an embodiment of the present invention according to the first aspect, the communication module 10 may be implemented as a network adaptor supporting a combination of the above-mentioned wire or wireless communication technologies depending on types of connections provided between the computer device 100 and any external devices mentioned in the present document.

Each urine spectrum received by the communication module 10 is transmitted via the communication bus 30 directly to the urine-analysis module 20 to allow the urine spectrum to be processed by the urine-analysis module 20. In another embodiment of the present invention, the urine spectrum and the personal patient data as received by the communication module 10 may be transmitted both via the communication bus 30 to the local storage 40 to be stored therein, and the urine-analysis module 20 may access the local storage 40 via the communication bus 30 to retrieve the previously stored urine spectra and the personal patient data so as to further process them. Thus, having received the first urine spectrum generated by the light detector 400, the second urine spectrum generated by the light detector 400 and the personal patient data, the urine-analysis module 20 shown in FIG. 1 combine the received urine spectra and the received personal patient data to generate a combined urine data array or a single urine data matrix. In a preferred example, data in the urine data matrix generated by the urine-analysis module 20 shown in FIG. 1 may be recorded as one string. In the embodiment of the present invention according to the first aspect where the light detector 400 shown in FIG. 1 is the 18-channel spectrometer, the urine data matrix generated by the urine-analysis module 20 shown in FIG. 1 may contain only one string and at least thirty two (32) columns, wherein a first part of the columns corresponds to the first light source 200, and a second part of the columns corresponds to the second light source 300, and a further third part of the columns corresponds to the personal patient data, wherein a number of columns in each of said first and second parts of the columns corresponds to the number of the spectrometer channels.

It is to note that the urine data matrix generated by the urine-analysis module 20 contains spectral data related to raw or original urine spectra generated by the light detector 400, i.e. the generated urine spectra are not pre-processed or filtered by the urine-analysis module 20 before generating the urine data matrix by combining the urine spectra with the personal patient data corresponding to the urine spectra. Thus, the urine-analysis module 20 requires less processing and memory resources to be used for generating the urine data matrix and adds less noises to the spectral data contained in the urine spectra, thereby resulting in less false determinations of urine parameters to be determined.

It is to further note that the personal patient data contained in the urine data matrix generated by the urine-analysis module 20 allow less false determinations of urine parameters to be determined by the urine-analysis module 20 or allow a reduced probability of incorrect determinations of urine parameters to be provided.

The urine-analysis module 20 shown in FIG. 1 is further configured to feed the generated urine data matrix to at least one computing model or a plurality of computing models, each computing model being based on a predetermined personal patient data (e.g. a patient height, weight, gender, age and/or diagnosis) and a predetermined correlation between a particular urine parameter obtained by a standard urine analysis method and measured urine spectra corresponding to the personal patient data, so as to determine at least one urine parameter or a plurality of urine parameters corresponding to the patient. In other words, applying, by means of the urine-analysis module 20 shown in FIG. 1, the pre-produced computing models to the generated urine data matrix allows particular urine parameters to be identified (i.e. determination on whether particular urine parameters are indicative for the analyzed urine or presented in the analyzed urine or determination on which of the predefined urine parameters are indicative for the analyzed urine or presented in the analyzed urine) or allows particular urine parameters to be evaluated (i.e. determination of values of particular urine parameters).

Thus, each of the computing models allows only corresponding one of the urine parameters to be determined within the urine analysis. It is to note that data on the computing models may be preliminarily stored in the local storage 40, and the urine-analysis module 20 may access the local storage 40 via the communication bus 30 to retrieve the previously stored data on the computing models to be used for determining urine parameters, each retrieved computing model corresponding to a particular one of the urine parameters to be determined/evaluated, wherein the computing models to be used by the urine-analysis module 20 may be pre-programmed or pre-configured by a user or defined by the user in any particular use of the system 1000.

The standard urine analysis method to be used in a corresponding one of the computing models applied by the urine-analysis module 20 to the fed urine data matrix depends on a particular urine parameter to be determined or evaluated with said computing model. In particular, the standard urine analysis method may be one of the following urine analysis methods known in the art: refractometry, dry chemistry, flow cytometry, microscopy and other appropriate urine analysis methods. For example, specific gravity (SG) may be measured by using an automated transmission refractometry method; pH, protein (PRO), bilirubin (BIL), glucose (GLU), ketones (KET), leukocyte esterase (LEU), nitrite (NIT), and urobilinogen (URO) may be determined by using a chemical analysis based on a dual-wavelength reflectance method; single wavelength reflectance for blood (BLD), red blood cells (RBC), white blood cells (WBC), squamous epithelial cells (SEC), hyaline casts (HC), bacteria (BACT), crystals (CRY), yeasts (YEA), transitional epithelial cells (TEC), pathological casts (PC), mucus (MUC) and spermatozoa (SPERM) may be determined by using a flow cytometry method.

The computing models used by the urine-analysis module 20 shown in FIG. 1 may be in the form of any appropriate pre-trained neural network, machine-learning model or any other appropriate preliminary produced computer model known in the art. In particular, each of the computing models used by the urine-analysis module 20 shown in FIG. 1 is preliminarily trained on a plurality of measured urine spectra and predetermined patient data corresponding to said measured urine spectra, wherein each urine spectrum of said measured urine spectra corresponds to a patient having a particular urine parameter determined by using a corresponding standard urine analysis method or a patient having a particular urine parameter with a value preliminarily measured by using a corresponding standard urine analysis method, and wherein said measured urine spectra correspond to patients having said urine parameter. Therefore, each of the computing models used by the urine-analysis module 20 shown in FIG. 1 is preliminarily trained to identify or evaluate a particular one of the urine parameters to be detected based on a combination of two different urine spectra related to the same patient urine sample and on predetermined patient data corresponding to said urine spectra.

In a preferred example, each of the computing models used by the urine-analysis module 20 for determining or evaluating urine parameters is a particular PLS regression model.

In particular, the urine-analysis module 20 may be further configured to retrieve or receive personal patient data (e.g. a patient height, weight, gender, age and/or diagnosis) from the local storage 40 via the communication bus 30, the received patient data corresponding to the first and second urine spectra received from the light detector 400, and to combine the received personal patient data together with the received first and second urine spectra in order to generate the urine data matrix to be fed to the computing models used by the urine-analysis module 20, so that the generated urine data matrix may further contain the received personal patient data or further based on the personal patient data (i.e. in addition to the received first and second urine spectra corresponding to the patient).

In another embodiment of the present invention, the personal patient data to be used by the urine-analysis module 20 for generating the urine data matrix correspond to a particular patient and may be stored in the data server 600, the cloud storage 700 or the external storage 800, so that the urine-analysis module 20 may be further configured to access said storage for retrieving or receiving the personal patient data (e.g. a patient height, weight, gender, age and/or diagnosis) corresponding to a particular patient and configured to combine the urine spectra received from the light detector 400 with the received personal patient data corresponding to the received urine spectra for generating the urine data matrix to be fed to predetermined computing models.

In a preferred embodiment of the present invention, the urine-analysis module 20 may be configured to apply each of the computing models stored in a data storage (e.g. the local storage 40) accessed by the urine-analysis module 20 to the generated urine data matrix by (i) categorizing the urine matrix according to predetermined target urine parameters (i.e. PRO, BIL, GLU, KET, NIT, URO, WBC, RBC, BLD, SEC, HC, BACT, CRY, YEA, TEC, PCT, MUC, etc.) into a category of molecular-scale biomarkers (PRO, BIL, GLU, KET, NIT, URO) and a category of cell-size biomarkers (e.g. WBC, RBC, BLD, SEC, HC, BACT, CRY, YEA, TEC, PCT, MUC) and by feeding the generated urine matrix to two PLS regression models, each corresponding to one of the determined urine parameter categories and being based on a predetermined correlation between particular urine parameters obtained by standard urine analysis methods and measured urine spectra, for determining said at least one urine parameter.

In other embodiments of the present invention, the urine-analysis module 20 may be configured to apply each of the computing models stored in a data storage (e.g. the local storage 40) accessed by the urine-analysis module 20 to the generated urine data matrix by (i) categorizing the urine matrix according to predetermined target urine parameters (i.e. PRO, BIL, GLU, KET, NIT, URO, WBC, RBC, BLD, SEC, HC, BACT, CRY, YEA, TEC, PCT, MUC, etc.) into at least two urine parameter category (e.g. at least (1) a category of molecular-scale biomarkers such as PRO, BIL, GLU, KET, NIT, URO and (2) a category of cell-size biomarkers (e.g. WBC, RBC, BLD, SEC, HC, BACT, CRY, YEA, TEC, PCT, MUC)) and by feeding the generated urine matrix to at least two PLS regression models (i.e. at least two PLS regression models), each corresponding to one of the determined urine parameter categories and being based on a predetermined correlation between particular urine parameters obtained by standard urine analysis methods and measured urine spectra, for determining said at least one urine parameter.

In various embodiments of the present invention, the urine-analysis module 20 may be further configured to preliminary normalize each of the first and second urine spectra received from the from the light detector 400 by maximum intensity before generating the urine data matrix. Thus, in such embodiments of the present invention, the generated urine data matrix to be fed to predetermined computing models is comprised or formed of a combination of the normalized first and second urine spectra and personal patient data (e.g. a patient height, weight, gender, age and/or diagnosis) corresponding to the said urine spectra. Thus, in such embodiments of the present invention, the normalization of the each of the first and second urine spectra before generating the urine data matrix does not allow amplitude variations arising from a cylindrical geometry of the urine container 500 or differences in urine volume in the urine container 500 to alter shape-based spectral features used for the urine data matrix.

In some embodiments of the present invention, each of the computing models used by the urine-analysis module 20 for determining the at least one urine parameter is preliminary trained by the urine-analysis module 20 by using a training dataset formed of urine sample spectra obtained by a standard spectrometer and synthetic spectra, each synthetic spectrum being formed by combining at least two of the obtained urine sample spectra related to the same urine parameter category determined by the urine-analysis module 20, wherein the training dataset and said synthetic spectra used by the urine-analysis module 20 are stored in a data storage (e.g. the local storage 40, the data server 600, the cloud storage 700 or the external storage 800) accessed by the urine-analysis module 20. It is to note that such preliminary training of the each of the computing models used by the urine-analysis module 20 for determining the at least one urine parameter allows minority classes to be rebalanced, while preserving composition-specific spectral signatures.

The urine-analysis module 20 and any other data-processing modules mentioned in the present document may be each implemented as a single processor, such as a common processor or a special-purpose processor (e.g., a digital signal processor, an application-specific integrated circuit, or the like). For example, the urine-analysis module 20 may be in the form of a central processing unit of the below-mentioned general-purpose computer (common computer) which may be the implementation of the computer device 100.

In some embodiments of the present invention, the communication module 10 in the computer device 100 may further communicatively connected to a packet capture device (not shown) in wire or wireless manner, in particular via the communication network 900. The packet capture device may be connected to the communication network 900 to capture data packets transmitted via the communication network 900 (network traffic) and to transmit the captured data packets to the communication module 10; the urine-analysis module 20 may further comprises a filtering or analyzing module (not shown) communicatively connected to the communication module 10 and the urine-analysis module 20 via the communication bus 30 to process the data packets received by the communication module 10. The analyzing module may be further configured or programmed to extract all files comprised in the data packets received from the communication module 10 and to analyze each of the extracted files to identify its format, wherein the analyzing module may be further configured or programmed to transmit each file having a format corresponding to a urine spectrum or urine spectra to the urine-analysis module 20 via the communication bus 30.

In various embodiments of the present invention, the computer device 100 may be in the form of a computing device comprised of a combination of a hardware and software or a general-purpose computer having a structure known for those skilled in the art. In an embodiment of the present invention, the computer device 100 may be implemented as a single computer server, such as «Dell™ PowerEdge™» server running the operating system «Ubuntu Server 18.04». In some embodiments of the present invention, the computer device 100 may be in the form of a table computer, laptop, netbook, smartphone, tablet and any other electronical or computing device appropriate for solving the above-mentioned prior art problems. In other embodiments of the present invention, the computer device 100 may be implemented in any other suitable hardware, software, and/or firmware, or a combination thereof. A particular implementation of the computer device 100 is not limited by the above-mentioned examples.

The local storage 40 stores executable program instructions or commands allowing the operation of functional modules integrated to the computer device 100 to be controlled, wherein said functional modules are the communication module 10, the urine-analysis module 20 and any other functional modules mentioned in the present document as a part of the computer device 100. Meanwhile, such executable program instructions or commands as stored in the local storage 40 also allow the functional modules of the computer device 100 to implement their functionalities. Furthermore, the local storage 40 stores different additional data used by the functional modules to provide their outputs.

The local storage 40 may be realized as a memory, a hard disk drive or any appropriate long-term storage. For example, the local storage 40 may be in the form of a data storage of the above-mentioned general-purpose computer which may be the implementation of the computer device 100.

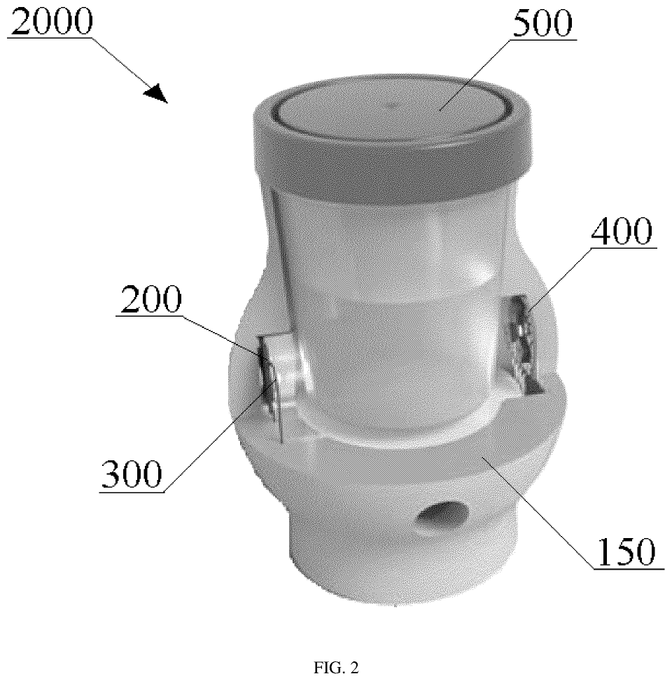

The first light source 200, the second light source 300 and the computer device 100 as described above for the first aspect of the present invention may be corresponding functional components of an integral device 2000 for analyzing urine (also referred to in the present document as a urine-analyzing device 2000). It is to note that the urine-analyzing device 2000 may be portable, hand-held or stationary. FIG. 2 is a block diagram illustrating the urine-analyzing device 2000 according to the second aspect of the present invention.

As shown in FIG. 2, the urine-analyzing device 2000 comprises a docking station or base 150 designed to receive the urine container 500, so that the urine container 500 preliminarily filled with a urine or a urine sample to be analyzed may be easily installed or mounted on the base 150 such that the urine container 500 is positioned between the light detector 400 and the light sources 200, 300. In an embodiment of the present invention, the urine container 500 shown in FIG. 2 may be releasably attached or secured to the base 150. In another embodiment of the present invention, the urine container 500 shown in FIG. 2 may be integral with the base 150.

Further, as shown in FIG. 2, the first light source 200 and the second light source 300 are each installed or mounted on the base 150 such that the light emitted by any of the light sources 200, 300 is directed to the urine container 500.

Further, as shown in FIG. 2, the light detector 400 is also installed or mounted on the base 150 such that the light transmitted or passed through urine or urine sample contained in the urine container 500 is received or detected by the light detector 400.

In the urine-analyzing device 2000 shown in FIG. 2, the computer device 100 (not shown in FIG. 2) is installed or mounted inside the base 150, wherein the computer device 100 may be formed as a processing device, processor or any other on-board computing device. It is to note that the operation of the light sources 200, 300 and the operation of the urine container 400 may be controlled by the computer device 100 or another on-board or external control device connected to said functional components in a wire or wire-less manner. The computer device 100 in the urine-analyzing device 2000 shown in FIG. 2 substantially performs the same functionalities as mentioned above for the system 1000, i.e. receives the first urine spectrum corresponding to the first light source 200 and the second urine spectrum corresponding to the second light source 300 from the light detector 400 and also receives personal patient data stored in a data storage accessed by the computer device 100 so as to combine the received first and second urine spectra with the received personal patient data corresponding to said urine spectra for producing or generating a urine data matrix and, then, feeds the generated urine data matrix to one or more computing models stored in a data storage accessed by the computer device 100, each computing model being based on predetermined personal patient data and a predetermined correlation between a particular urine parameter obtained by a standard urine analysis method and measured urine spectra corresponding to the personal patient data, so as to determine at least one urine parameter (i.e. allows identification and evaluation of each of the required urine parameters).

Submitted in the below table are experiment results obtained for four different computing models for four different datasets, each dataset being used by the computer device 100 for generating the urine data matrix to be fed to each of said computing models. In particular, the computing models used in the experiment are as follows: 1. a computing model based on a Random Forest Classifier (RFC); 2. a computing model based on a Gradient Boosting Classifier (GBC); 3. a computing model based on a Multi-Layer Perceptron Classifier (MLPC); and 4. a computing model based on a Partial Least-Squares Classifier (PLSC). Also, datasets processed by the computer device 100 for generating the urine data matrix to be fed to each of the computing models are as follows: 1. Urine spectra only; 2. Urine spectra in combination with personal patient data of first type (namely, patient age); 3. Urine spectra in combination with personal patient data of second type (namely, patient gender); and 4. Urine spectra in combination with the personal patient data of first type (namely, patient age) and the personal patient data of second type (namely, patient gender), wherein urine spectra as used in different datasets in a raw form (i.e. original form without any pre-processing procedures applied thereto) are the same, and personal patient data of the same type as used in different datasets are the same.

It is to note that the above-mentioned computing models (i.e. four different machine learning algorithms used in the experiment) may be compared with standard methods by using universal quality metrics which would work for any datasets in medical tests. Please note that F_score validation, sensitivity validation and specificity validation as presented in corresponding columns of the below Table are quality metrics of a particular computing model (a particular machine learning algorithm) for a particular dataset as obtained during the validation process of the developed machine learning algorithms.

In particular, the sensitivity is determined by using true-positive (TP) and false-negative (FN) rates and represents the probability of a positive test among patients with a disease:

$$SENS = \frac{TP}{TP + FN} . \qquad ( 1 )$$

Also, the specificity is defined by using true-negative (TN) and false-positive (FP) rates and represents the probability of negative test results among patients without a disease (a true negative rate):

$$SPEC = \frac{TN}{TN + FP} . \qquad ( 2 )$$

The sensitivity and the specificity are quality metrics having an explicit probabilistic interpretation.

However, the F-score is a quality metric combining the sensitivity and the specificity, so that the F-score is the most appropriate quality metric for choosing the best of the computing models under examination (i.e. the best machine learning algorithm among the examined machine learning algorithms). In particular, the F-score is equal to a weighted harmonic mean of sensitivity and specificity:

$${F}_{β} - score = ( 1 + {β}^{2} ) \cdot \frac{SPEC \cdot SENS}{( {β}^{2} \cdot SPEC ) + SENS} , \qquad ( 3 )$$

wherein: β=1 for obtaining a balanced sensitivity and specificity.

The below table 1 presents the sensitivity, specificity and F-score metrics obtained during the validation process of the developed machine learning algorithms.

All the machine learning algorithms used in the experiment have been built to detect bacteria in urine contained in the urine container according to the present invention, wherein reference data have been obtained by using the automated UX-2000 analyzer (Sysmex Corp., Japan), and dichotomized laboratory analyses have been considered based on the following cut-off values for bacteria: 300/μL.

TABLE 1
Experiment results for different computing models

| | | | | Computing Model/ | |
|---|---|---|---|---|---|
| | F_score | Sensitivity | Specificity | Machine Learning | |
| No. | validation | validation | validation | Algorithm | Dataset |
| 0 | 0.763 | 0.700 | 0.857 | RFC | Spectra |
| 1 | 0.763 | 0.708 | 0.848 | RFC | Spectra + Age |
| 2 | 0.740 | 0.658 | 0.870 | RFC | Spectra + Gender |
| 3 | 0.756 | 0.692 | 0.855 | RFC | Spectra + |
| | | | | | Gender + |
| | | | | | Age |
| 4 | 0.743 | 0.646 | 0.904 | GBC | Spectra |
| 5 | 0.764 | 0.709 | 0.856 | GBC | Spectra + Age |
| 6 | 0.746 | 0.648 | 0.905 | GBC | Spectra + Gender |
| 7 | 0.772 | 0.707 | 0.877 | GBC | Spectra + |
| | | | | | Gender + |
| | | | | | Age |
| 8 | 0.709 | 0.674 | 0.763 | MLPC | Spectra |
| 9 | 0.703 | 0.631 | 0.807 | MLPC | Spectra + Age |
| 10 | 0.665 | 0.597 | 0.786 | MLPC | Spectra + Gender |
| 11 | 0.606 | 0.570 | 0.654 | MLPC | Spectra + |
| | | | | | Gender + |
| | | | | | Age |
| 12 | 0.824 | 0.783 | 0.876 | PLSC | Spectra |
| 13 | 0.823 | 0.769 | 0.887 | PLSC | Spectra + Age |
| 14 | 0.841 | 0.810 | 0.884 | PLSC | Spectra + Gender |
| 15 | 0.838 | 0.804 | 0.880 | PLSC | Spectra + |
| | | | | | Gender + |
| | | | | | Age |

As follows from the above Table, results experimentally obtained for the computing model based on the Gradient Boosting Classifier and the computing model based on the Partial Least-Squares Classifier generally illustrate the improvement of the quality metrics in case when the personal patient data are further used together with the urine spectra within the urine data matrix generated by the computer device 100, the generated urine data matrix being fed to any of the computing models.

In an embodiment of the present invention, a diffuser (not shown) in the urine-analyzing system 1000 shown in FIG. 1 or the urine-analyzing device 2000 shown in FIG. 2 may be installed or mounted on the base 150 between the urine container 500 and the light detector 400 (i.e. positioned in front of a sensing region of the light detector 400), wherein the diffuser is designed to scatter or homogenize the light passed through urine in the urine container 500. The diffuser may be either a volumetric type (e.g., a translucent volume of scattering material) or a surface diffuser (e.g., a roughened or patterned plate) that randomizes or homogenizes incoming light. The diffuser provides the most observable positive effect in case when the urine container 500 is cylindrically shaped or does not have flat parallel walls. In particular, when the urine container 500 having a cylindrical form (e.g., the urine container 500 formed as a standard urine sample cup) is partially or fully filled with urine and placed in the sensing region of the light detector 400, it effectively forms a cylindrical lens. Meanwhile, light emitted in the direction of the urine container 500 passes through curved surfaces of the urine container 500 and experiences additional focusing and/or deflection, wherein such negative effects may change from one placement of the urine container 500 on the base 150 to another placement of the urine container 500 on the base 150, especially they may change in case when the urine container 500 is not inserted at a perfectly consistent angle. Furthermore, there may be mechanical play or “slack” (i.e. a backlash) in a holder used in the base (150) for holding the urine container 500, so that slight shifts in orientation cause varying refraction and scattering of the transmitted light. In a laboratory setting, spectroscopic measurements of liquids (e.g. urine) typically employ cuvettes having flat parallel walls to avoid any lensing effect. By contrast, using common cylindrical containers is far more convenient and cost-effective in many real-world applications, but it introduces a potential for orientation-dependent spectral distortions. Although empirical data indicate that small misalignments do not drastically reduce predictive accuracy on a well-characterized “master” device (i.e., the device used to build or train the classification model), replicating the same performance on additional (“slave”) devices often reveals discrepancies. These differences may be amplified by slight mechanical tolerances, differences in optical alignment, or variations in container insertion. However, the diffuser reduces orientation-induced spectral distortions caused by the urine container 500 having a cylindrical wall provided with non-planar entry and exit surfaces for the transmitted light. In particular, to mitigate the unpredictable focusing effect of the cylindrical urine container 500, the diffuser needs to be placed before the light detector 400. The rationale is that randomized scattering of the transmitted beam helps unify the detected light distribution, reducing sensitivity to small angular offsets introduced by the container's orientation. Surprisingly, it was found that once the diffuser is in place, repeated insertions of the same cylindrical container yield spectra that match much more closely than when no diffuser is used. As a result, subsequent steps-such as calibrating a secondary (“slave”) device to match the “master” device-become simpler and more reliable.

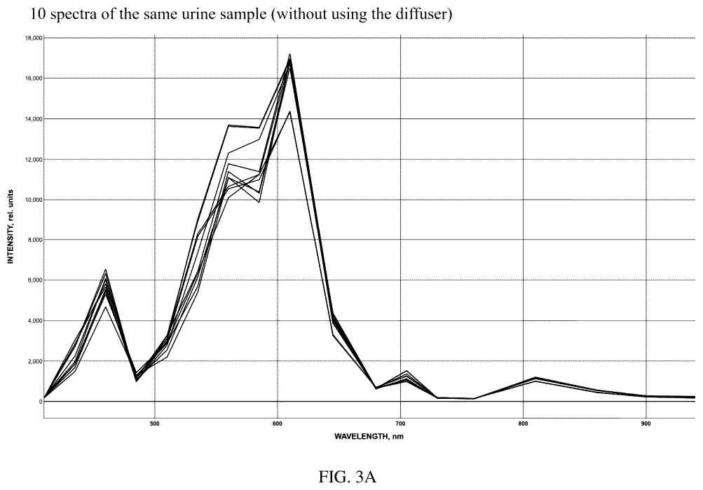

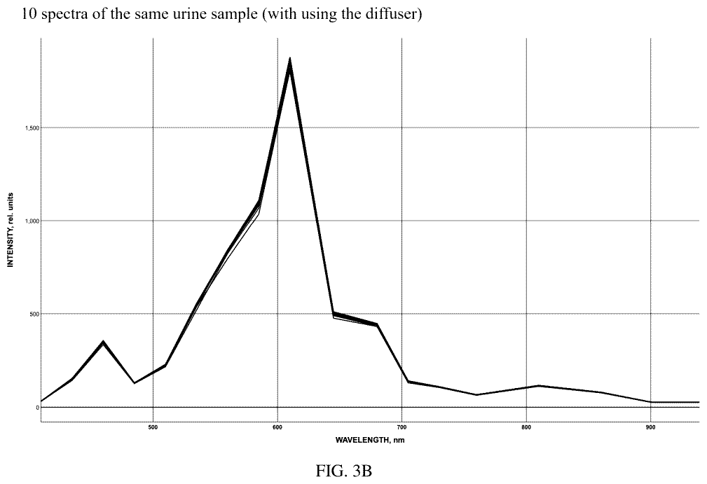

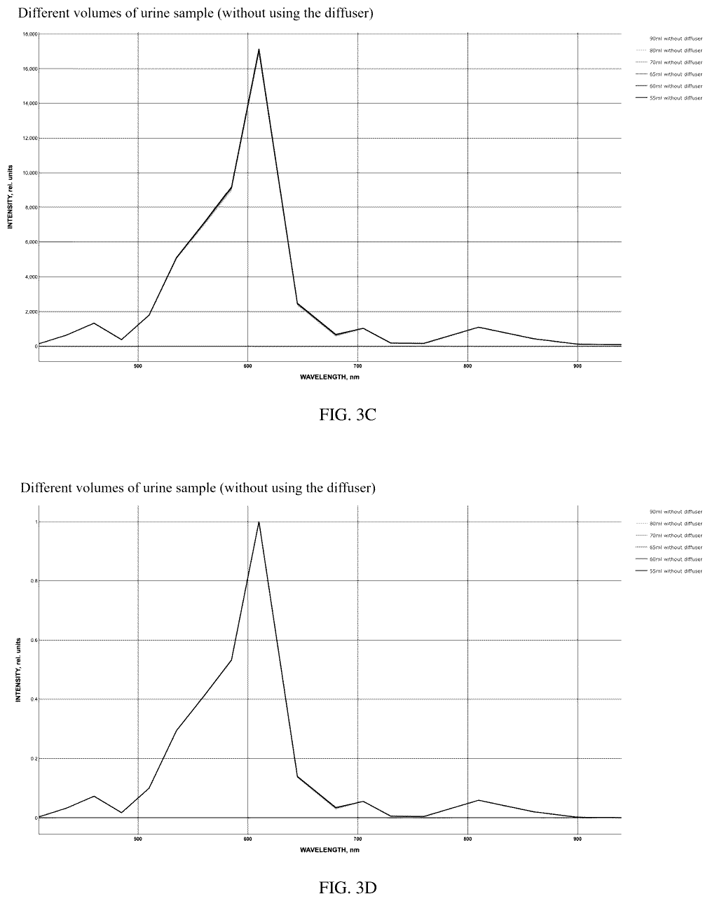

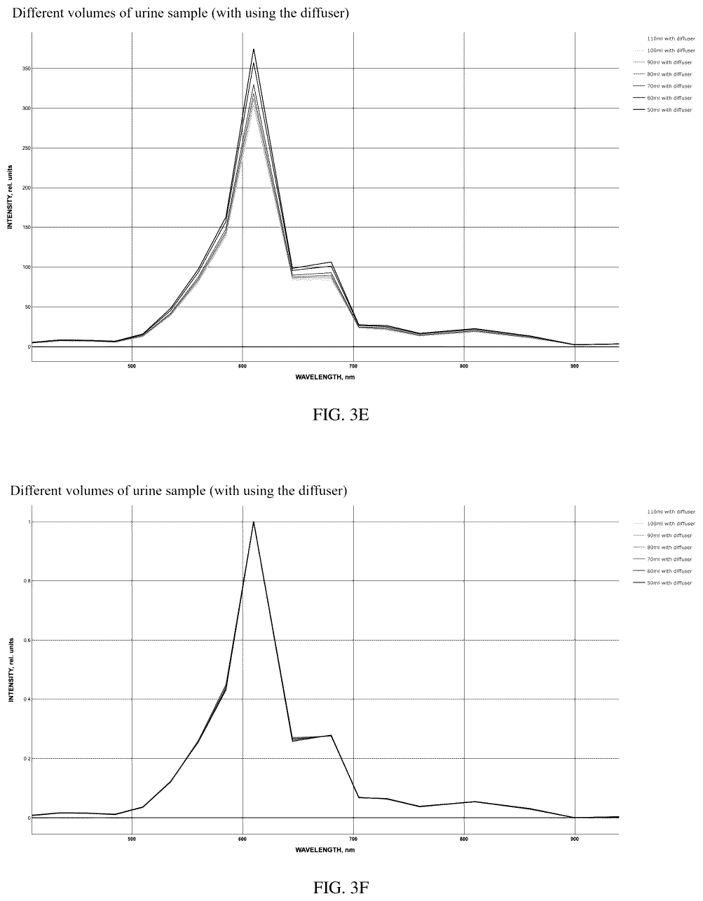

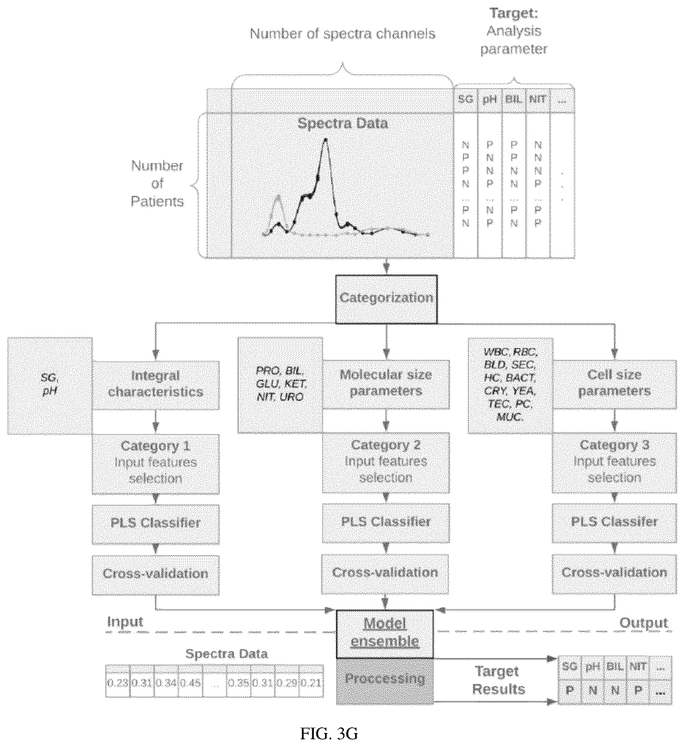

As shown in FIG. 3A and FIG. 3B, a difference between performing multiple measurements on the same urine sample without the diffuser placed between the urine container 500 and the light detector 400 (see FIG. 3A) and with the diffuser placed between the urine container 500 and the light detector 400 (see FIG. 3B) can be observed. FIG. 3A shows significant variation in intensity across repeated spectra due to slight orientation or insertion angle changes of the cylindrical urine container 500. In contrast, FIG. 3B illustrates that once the diffuser is added, the measured spectra remain closely aligned, thereby demonstrating the diffuser's capacity to scatter or homogenize the transmitted light and reduce orientation-induced variability.

In embodiments of the present invention where the diffuser is installed between the urine container 500 and the light detector 400, unexpected synergy between the diffuser and the urine container 500 is revealed. A person skilled in conventional spectroscopy might assume it is simpler or more standard to redesign the urine container 500 to have flat walls. However, for practical or economic reasons, such modifications may be impossible. It is likewise not obvious that the inserting of the diffuser downstream from the urine container 500 (near the light detector 400) would sufficiently homogenize the beam and allow consistent readings regardless of a container tilt. In fact, one might fear that scattering introduced by a diffuser would compromise spectral resolution. Yet experiments revealed the contrary—that by carefully selecting or designing a volumetric or surface diffuser, the device can reduce orientation sensitivity to negligible levels, without significantly degrading the informative spectral features.

In embodiments of the present invention where the diffuser is installed between the urine container 500 and the light detector 400, it becomes feasible to find a single or set of linear transformations that map “slave” device spectra to the “master” device's spectral domain. In practice, such calibration can be established by measuring several known reference spectra, for instance:
A real or synthetic urine sample with known spectral characteristics;
Distilled water as a baseline;
An empty cylindrical container to capture background scattering;
Direct illumination spectra measured (for instance, removing the urine container 500 and measuring the source's raw emission);
Other standard reference liquids that exhibit well-characterized optical features in the relevant spectral bands.

By measuring each reference on both the master device and the slave device, one can derive transformations—e.g., a linear scaling plus offset per channel—that align the two sets of spectra. Because the diffuser minimizes random orientation-related differences, these transformations apply robustly across multiple test samples. Consequently, the model predictions from the slave device can be matched closely to those from the master device, improving reproducibility and overall reliability in a multi-device environment.

In embodiments of the present invention where the diffuser is installed between the urine container 500 and the light detector 400, the diffuser-based hardware scheme can be integrated seamlessly with the machine-learning approach previously described (for instance, partial least squares classification, multi-category grouping, synthetic data augmentation, etc.). After the diffuser is introduced and the device is calibrated, training data can be collected with minimal distortion from container orientation. Alternatively, if the “master” data was collected without a diffuser, the slave device can incorporate one, with an appropriate calibration procedure so that final predicted parameters remain consistent with the master device's reference model. Such synergy of hardware and software solutions ensures that the system can handle wide variations in urine containers and device assemblies, while still producing reliable predictions of urine parameters.

Given the complexities of cylindrical urine container 500 insertion and the potential alternatives (like redesigning the container or imposing strict mechanical constraints), it is by no means obvious that adding a diffuser in front of the detector is the straightforward fix for normalizing orientation effects. However, extensive experimentation has shown that this approach indeed yields a simple, low-cost, and robust solution, clearly meeting industrial applicability requirements for a portable or clinical urine analysis system.

In other embodiments of the present invention according to the first or second aspect, an unexpected optical behavior emerges when the diffuser is placed between the urine container 500 and the light detector 400. In particular, empirical observations show that, when more liquid of identical composition is added—thus increasing the fluid volume above the detector's field of view—the measured spectral amplitude decreases (i.e., intensity is lower in each channel), yet the overall shape of the spectral curve (i.e., the ratio of intensities across wavelengths) remains remarkably constant. By applying a straightforward normalization step—such as dividing each measured channel by the maximum intensity in that spectrum—these spectra for different fluid volumes become nearly identical. A person skilled in the art might initially assume that adding a diffuser in front of the detector would degrade spectral resolution or exacerbate any dependence on fill level, because additional scattering could entangle the light's path through the container. Equally, one might expect that once the detector's field of view is submerged, the fluid volume above that level would have no particular effect on the measured spectrum. Experimental results with the present invention, however, reveal a distinct phenomenon:

### 1. Without Using the Diffuser

Adding extra fluid of the same composition (within the same cylindrical container) does not measurably change the amplitude or shape of the optical spectrum, suggesting that increasing fluid volume is optically inconsequential in that configuration. As shown in FIG. 3C (without the diffuser, multiple volumes of urine sample), the absolute intensities can spike to very high counts (e.g., 16,000-18,000 near the main peak). Volume changes between 90 mL, 80 mL, and 70 mL do not significantly shift overall amplitude or shape; thus, subtle fill-level differences might remain undetected unless further calibration is performed. As shown in FIG. 3D (without the diffuser, multiple volumes of urine sample; normalized spectra), similar trends appear, though certain curves reveal a small shift in peak amplitude. The main takeaway is that the container's geometry, without a diffuser, does not yield a predictable amplitude reduction as volume increases.

### 2. Use of the Diffuser

Inserting the diffuser unexpectedly induces amplitude changes with increasing fluid volume, while preserving the underlying spectral shape. That is, each wavelength channel's intensity decreases in a consistent manner, yet the ratio of intensities (the shape) remains effectively the same for the same fluid composition. As shown in FIG. 3E (without the diffuser, multiple volumes of urine sample), here the maximum amplitude is far lower, e.g., under 350. Each line's amplitude decreases as the fluid volume increases (from 50 mL to 110 mL), but the shape (peak position and relative slope) remains the same. This phenomenon can be exploited for more robust calibration, as amplitude-level differences are uniform across the spectrum. As shown in FIG. 3F (without the diffuser, multiple volumes of urine sample; normalized spectra), the same sample exhibits consistently shaped spectra. The difference in intensities is largely a linear factor, so normalizing each curve to its peak yields nearly perfect overlap.

### 3. Use of Normalization Restoring the Overlap

Because the shape is preserved, an amplitude-based normalization (e.g., division by the maximum channel, area, or reference channel) ensures that all the resulting normalized urine spectra coincide for the same urine composition, independent of how much urine is added.

### Benefits for Calibration and Machine-Learning Classification

The synergy of diffuser placement and simple signal normalization reduces or eliminates adverse effects of fluid-level changes on the classification or parameter detection process, particularly when machine-learning (ML) algorithms are employed. For instance:
Robustness Against Over/Under-Filling: The user can fill a standard cylindrical container to varying levels without compromising the shape-based spectral features used by partial least squares (PLS) or other ML classification models.
Consistent Device Calibration: Even if the amplitude changes with additional fluid, normalizing the measured spectrum ensures stable shape-based signatures. Devices can be calibrated once to handle a broad range of fill levels, facilitating a consistent interpretation of fluid composition.
Preservation of Shape: Since shape invariance (rather than amplitude) is primarily used by the ML model to identify or quantify parameters (e.g., specific urine biomarkers), the device remains independent of small or moderate variations in fluid volume in practice.

Overall, by combining the diffuser placed in front of the light detector 400 with a simple post-processing normalization step, the invention achieves high reproducibility and reliability in analyzing liquids. This approach runs contrary to typical assumptions that a diffuser either (i) has minimal effect if fluid volume is above the line of sight or (ii) disrupts measurement accuracy by introducing scattering. Instead, the invention leverages the fact that amplitude alone can vary consistently without affecting the composition-relevant shape of the spectrum.

### Multi-Stage Machine-Learning Procedure (Parameter Categorization+PLS+Grouped Cross-Validation)

In order to improve the accuracy and robustness of identifying specific urine parameters (or detecting particular diseases) based on transmission spectra, the present invention, in certain embodiments, deploys a multi-stage machine-learning (ML) procedure that incorporates (i) a preliminary categorization of parameters, (ii) selection of optimized input features, and (iii) a specialized training method using partial least squares (PLS) classification together with cross-validation. A representative workflow is depicted in FIG. 3G. In particular, FIG. 3G illustrates how the computer device 100 processes the measured urine spectra, categorizes said urine spectra into categories of urine parameters to be predicted, selects features from the urine spectra, and then applies the PLS classification for each category of parameters.

### Preliminary Parameter Categorization

In an exemplary embodiment, each parameter measured or predicted from the urine sample is preliminarily assigned to one of several categories (e.g., “integral characteristics,” “molecular size parameters,” and “cell size parameters”). Integral characteristics may include, for example, specific gravity (SG) and pH. Molecular size parameters may comprise protein, bilirubin, glucose, ketones, nitrite, urobilinogen, or other biomarkers of similar molecular scale. Cell size parameters might include blood cells (RBC, WBC), bacteria, pathological casts, crystals, yeast, and so forth. By distinguishing parameters in this manner, the computer device 100 can internally group urine spectra that are known to carry common optical or scattering signatures, thereby improving the training of the classification model.

### Partial Least Squares Classification

After assigning each parameter to one of these categories, a separate PLS-based classification model (or a multi-output classification structure using partial least squares regression) is trained for that particular category. Partial least squares are well suited to situations involving high collinearity among spectral channels (for instance, when there are multiple overlapping wavelength regions) and relatively small training sets. By focusing on a subset of conceptually related parameters in each PLS model, the computer device 100 can identify latent spectral components that correlate most strongly with the presence (or absence) of the urine parameter of interest.

### Training and Cross-Validation

To ensure robust model generalization and to fine-tune hyperparameters, the training stage can employ a stratified cross-validation scheme grouped by patient. “Stratification” ensures that positive/negative samples of each urine parameter are balanced between folds, while “grouping by patient” ensures that the same patient's urine spectra do not appear in both the training and validation subsets. This mitigates data leakage in cases of repeated measurements from a single patient.

Moreover, to optimize classification thresholds (i.e., probability cutoffs) and the number of latent factors for the PLS classifier, an exhaustive or grid search is performed. In one embodiment, the computer device 100 iterates over possible thresholds (from 0.0 to 1.0 in small increments) and over a range of PLS components (e.g., 2 through 30), measuring each configuration's sensitivity, specificity, or a combined metric such as the F_β-score. The computer device 100 then selects the threshold and number of PLS components that yield the best harmonic balance between sensitivity and specificity for that parameter category. In certain embodiments, the computer device 100 repeats the cross-validation procedure multiple times (for example, fivefold or sixfold repeated cross-validation) to achieve stable estimates of classification performance metrics. The final classification model is thus the result of a carefully optimized process using both parameter grouping (categorization) and PLS classification with systematically chosen hyperparameters.

### Overall Workflow

As shown in FIG. 3G, once the raw transmission spectra are captured by the light detector 400 (for example, from two light sources 200, 300 operating in different spectral ranges), the computer device 100 aggregates these spectral measurements into a spectral data array (or a matrix). The computer device 100 then performs the following operations:
1. Categorization—Each target urine parameter is assigned to a relevant category (e.g., integral, molecular, or cell-size parameters).
2. Feature Selection—Optionally, the computer device 100 can select only those spectral channels most indicative of the category of interest, filtering out channels with negligible predictive power.
3. PLS Classifier Training—A partial least squares classification step is executed for each category, adjusting internal model weights using a training procedure that incorporates cross-validation grouped by patient.
4. Threshold/Hyperspace Optimization—The classification threshold and the number of PLS components (latent factors) are systematically varied to maximize an F-score or other relevant metric.
5. Ensemble Integration—If multiple category-specific classifiers are used, the final output merges their predictions, yielding the presence or absence of each parameter (or disease) as a binary label.

This procedure has been shown to be industrially applicable in real-world clinical or personal health scenarios, as it offers a reliable, non-invasive methodology for diagnosing or screening various urine parameters using an economical optical device. The approach is not obvious from existing conventional spectroscopic or colorimetric methods since it exploits (i) the synergy of grouping heterogeneous parameters by physical scale, (ii) partial least squares classification in a multi-parameter context, and (iii) advanced cross-validation with grouped stratification. By combining these elements, the invention addresses the longstanding problem of reliably mapping multichannel transmission spectra to multiple urine parameters with improved accuracy and generalization.

In order to enhance the robustness and coverage of machine-learning models employed to classify or evaluate urine parameters from optical transmission spectra, certain embodiments of the present invention may incorporate a technique of synthetic data generation. Such synthetic data may supplement measured urine spectra with artificially created spectra, thereby enlarging the overall dataset and rebalancing underrepresented classes.

In an exemplary embodiment, an initial dataset of urine transmission spectra is collected from a plurality of patients, each spectrum being associated with at least one known target value (e.g., presence or absence of specific biomarkers). These urine spectra typically originate from a device comprising multiple light sources, for instance various LEDs covering visible and near-infrared ranges, or optionally including a fluorescence channel. Pre-processing steps (e.g., background subtraction) are performed to isolate meaningful spectral information. Consequently, each measured (real) spectrum may be represented as a multidimensional array of 54 or more channels, depending on the device configuration.

It is to note that synthetic spectra may be created by using the following methodology:

(a) Identifying Minor vs. Major Classes: the computer device 100 according to certain embodiments of the present invention may detect parameters or biomarkers that exhibit relatively few positive (or “anomalous”) samples as compared to negative (or “normal”) samples. Such imbalance can degrade the performance of typical supervised learning approaches.

(b) Selecting Candidate Spectra: for each minority category of interest (e.g., “anomalous” parameter values), all corresponding measured urine spectra may be grouped. The computer device 100 then selects pairs or tuples of those spectra and, through a predetermined combination rule, merges them into one or more synthetic samples. In certain embodiments, the combination rule is linear (e.g., averaging or weighted summation), though non-linear operations (e.g., polynomial or exponential mixing) can be employed to highlight relevant spectral signatures.

(c) Maintaining or Adjusting Ratios: the computer device 100 may iteratively generate additional synthetic samples to match or control the ratio of anomalous vs. normal classes, ensuring that the final augmented dataset reflects user-defined targets. Analogously, urine spectra belonging to the normal class may also be combined if needed to maintain distributional balance.

(d) Preserving Spectral Realism: in order to avoid excessive deviation from physically plausible spectra, the computer device 100 may enforce constraints (e.g., bounding amplitude in each channel, removing out-of-bounds outliers) so that the resultant synthetic spectra retain or accentuate only those variations consistent with measured urine samples.

As described below in details, an augmented dataset may be used in a training procedure in the following manner:

(a) After generating synthetic data, the computer device 100 may execute machine-learning training upon both the original (measured) data and the newly created augmented data. In certain embodiments, partial least squares (PLS) classification is applied. Cross-validation-preferably stratified by target categories and grouped by patient identifier-helps to mitigate overfitting and ensures that spectra from a single patient are not repeated in both training and validation sets.

(b) During each fold of cross-validation, the model's internal parameters (e.g., number of latent components in PLS) and classification thresholds (e.g., an F_β-score cutoff) are systematically tuned to maximize overall performance, specifically for multi-parameter or multi-biomarker classification.

(c) In some embodiments, training is repeated on the augmented dataset while validation is conducted upon the original (unaugmented) set of urine spectra. This procedure confirms whether the introduction of synthetic spectra improves generalization rather than introducing artificial biases.

In certain embodiments of the present invention, the synergy of (i) target-aware synthetic spectrum creation and (ii) PLS-based classification with grouped cross-validation proves advantageous. Traditional data augmentation is known in fields such as image processing; however, adapting such techniques effectively to multi-channel urine transmission spectra—where the task is to detect subtle absorption, scattering, or fluorescence differences—remains non-trivial. The above-described approach may especially benefit minority biomarkers that infrequently occur in the clinical dataset (e.g., Bilirubin or Urobilinogen).

As a specific, non-limiting example, Table 2 below illustrates F-score results for selected urine parameters upon validation when synthetic data are used, compared to the baseline without synthetic augmentation.

TABLE 2
Validation F-score Comparison for Selected Urine Parameters

| | Optical spectra | Validation | Validation | |
|---|---|---|---|---|
| | in a syn- | F-score | F-score | ΔF (Syn. − |
| Parameter | thetic dataset | (Synthetic) | (Original) | Orig.) |
| Urine pH | 8607 | 0.83 | 0.62 | +0.21 |
| Urine Specific | 71507 | 0.72 | 0.68 | +0.04 |
| Gravity | | | | |
| Glucose | 15063 | 0.70 | 0.55 | +0.15 |
| Casts | 38686 | 0.86 | 0.73 | +0.13 |
| Bilirubin | 2108 | 0.96 | 0.58 | +0.38 |

As follows from the above Table 2, certain parameters—particularly those with severe class imbalances (like Bilirubin at ˜1% positives)—exhibit marked gains in validation F-score (e.g., +0.38). Meanwhile, parameters with moderate fractions (Urine pH >7, Glucose, Casts) demonstrate improvements of +0.13 to +0.21 in the F-score metric. By carefully generating synthetic spectra that preserve or emphasize real spectral features, the invention thus addresses the longstanding problem of incomplete data coverage in diagnostic classification tasks.

In certain examples of the present invention, the original dataset may contain, for example, 849 measured urine spectra. The synthetic augmentation may increase the minority classes for Bilirubin, Urine pH >7, or other underrepresented anomalies. As a result, the final augmented dataset can exceed 100,000 urine spectra in total.

By ensuring that minority biomarkers are adequately represented, the present invention improves reliability in automated urine testing systems and personal healthcare devices, thereby aligning with key requirements of modern clinical analyzers.

The synergy between partial least squares classification, grouped cross-validation, and realism-preserving synthetic generation is neither obvious nor trivial from known practices in spectroscopic or machine-learning fields. Rather, it leverages subtle aspects of multi-channel urine data to yield robust classification benefits.

In an alternative embodiment of the present invention according to the first aspect or second aspect, each of the computing models used by the computer device 100 and provided with the urine data matrix previously generated by the computer device 100 may be alternatively based on predetermined personal patient data and a predetermined correlation between a particular disease diagnosed by a medical specialist or a standard medical diagnostic technique and measured urine spectra, so that the computer device 100 may allow at least one disease of the patient or a plurality of diseases of the patient to be revealed or determined (i.e. allows detection of each of the required patient diseases). In other words, the computer device 100 according to the present alternative embodiment of the present invention substantially generally performs the most functionalities mentioned above for the system 1000, i.e. the computer device 100 also receives the first urine spectrum corresponding to the first light source 200 and the second urine spectrum corresponding to the second light source 300 from the light detector 400 so as to combine them with predetermined personal patient data corresponding to the received first and second urine spectra for producing or generating the urine data matrix, however the above alternative computing models based on predetermined personal patient data and a predetermined correlation between a particular disease diagnosed by a medical specialist or a standard medical diagnostic technique and measured urine spectra are applied to the generated urine data matrix, and patient diseases (i.e. not the at least one urine parameters related to the patient) are alternatively determined or detected as a result or as an output provided by the computer device 100. Thus, in the present alternative embodiment of the present invention according to the first or second aspect, each of the computing models allows only corresponding one of the patient diseases to be determined within the urine analysis. Meanwhile, each of the computing models used by the urine-analysis module 20 contained in the computer device 100 according to the present alternative embodiment of the present invention is preliminarily trained on a plurality of measured urine spectra and predetermined patient data corresponding to said measured urine spectra, wherein each urine spectrum of said measured urine spectra corresponds to a patient having a particular diagnosed disease, and wherein said spectra correspond to patients having said diagnosed disease. Therefore, each of the computing models used by the urine-analysis module 20 contained in the computer device 100 according to the present alternative embodiment of the present invention is preliminarily trained to determine or detect a particular one of the patient diseases to be detected based on a combination of two different urine spectra related to the same patient urine sample and on predetermined personal patient data corresponding to said urine spectra. It is clear for a skilled person that the computer device 100 in present alternative embodiment of the present invention according to the first aspect may be used as the above-described functional component of the system 1000 shown in FIG. 1 or used as a processing device in the urine-analyzing device 2000 shown in FIG. 2. It is to note that the present alternative embodiment of the present invention according to the first aspect or second aspect actually discloses a system for analyzing urine according to a third aspect of the present invention and a urine-analyzing device according to a fourth aspect of the present invention.

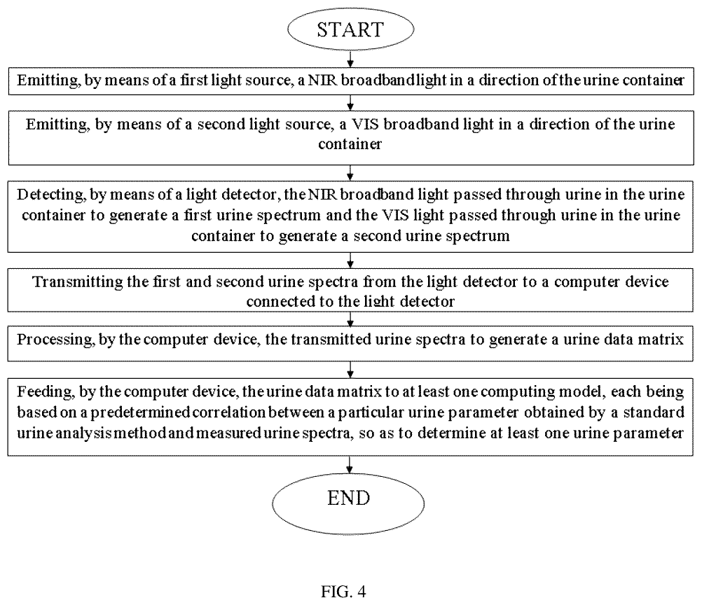

FIG. 4 illustrates a flow diagram of a method of analyzing a urine according to a fifth aspect of the present invention.

The method of FIG. 4 may be implemented by the above system 1000 according to the first aspect of the present invention as shown in FIG. 1 or the above urine-analyzing device 2000 according to the second aspect of the present invention as shown in FIG. 2. Anyway, the method of FIG. 4 may be implemented by any computing or electronic device known in the art, in particular by a processing unit of the above-mentioned general-purpose computer.

The method of FIG. 4 comprises the following stages or steps:
(1) emitting, by means of a first light source, a NIR broadband light in a direction of the urine container;
(2) emitting, by means of a second light source, a VIS broadband light in a direction of the urine container;
(3) detecting, by means of a light detector, the NIR broadband light passed through urine in the urine container to generate a first urine spectrum and the VIS light passed through urine in the urine container to generate a second urine spectrum;
(4) transmitting the first and second urine spectra from the light detector to a computer device connected to the light detector;
(5) receiving, by the computer device, personal patient data corresponding to the transmitted urine spectra;
(6) combining, by the computer device, the transmitted urine spectra with the received personal patient data to generate a urine data matrix; and
(7) feeding, by the computer device, the generated urine data matrix to one or more computing models stored in a local storage accessed by the computer device, each computing model being based on predetermined personal patient data (e.g. a patient height, weight, gender, age and/or diagnosis) and a predetermined correlation between a particular urine parameter obtained by a standard urine analysis method and measured urine spectra corresponding to the personal patient data, so as to determine at least one urine parameter.

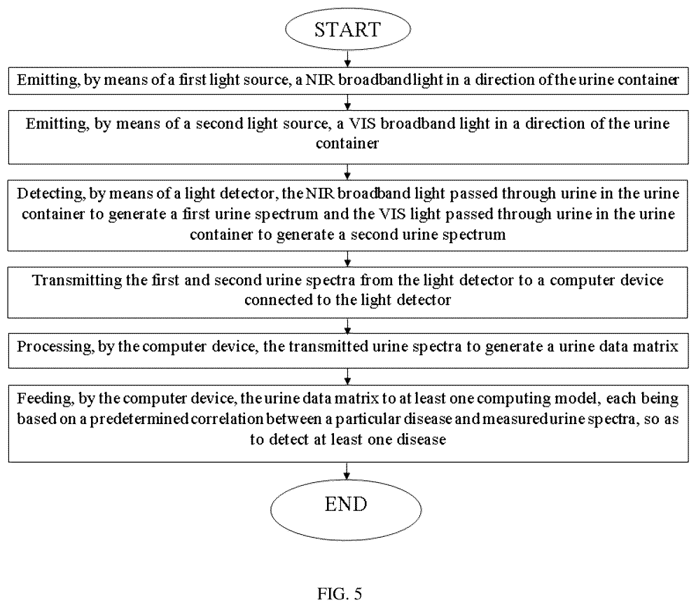

FIG. 5 illustrates a flow diagram of a method of analyzing a urine according to a sixth aspect of the present invention.

The method of FIG. 5 may be implemented by the above urine-analyzing system according to the third aspect of the present invention or the above urine-analyzing device according to the fourth aspect of the present invention. Anyway, the method of FIG. 5 may be also implemented by any computing or electronic device known in the art, in particular by a processing unit of the above-mentioned general-purpose computer.

The method of FIG. 5 comprises the following stages or steps:
(1) emitting, by means of a first light source, a NIR broadband light in a direction of the urine container;
(2) emitting, by means of a second light source, a VIS broadband light in a direction of the urine container;
(3) detecting, by means of a light detector, the NIR broadband light passed through urine in the urine container to generate a first urine spectrum and the VIS light passed through urine in the urine container to generate a second urine spectrum;
(4) transmitting the first and second urine spectra from the light detector to a computer device connected to the light detector;
(5) receiving, by the computer device, personal patient data corresponding to the transmitted urine spectra;
(6) combining, by the computer device, the transmitted urine spectra with the received personal patient data to generate a urine data matrix; and
(7) feeding, by the computer device, the urine data matrix to one or more computing models stored in a local storage accessed by the computer device, each computing model being based on predetermined personal patient data and a predetermined correlation between a particular disease and measured urine spectra corresponding to the personal patient data, so as to detect at least one disease.

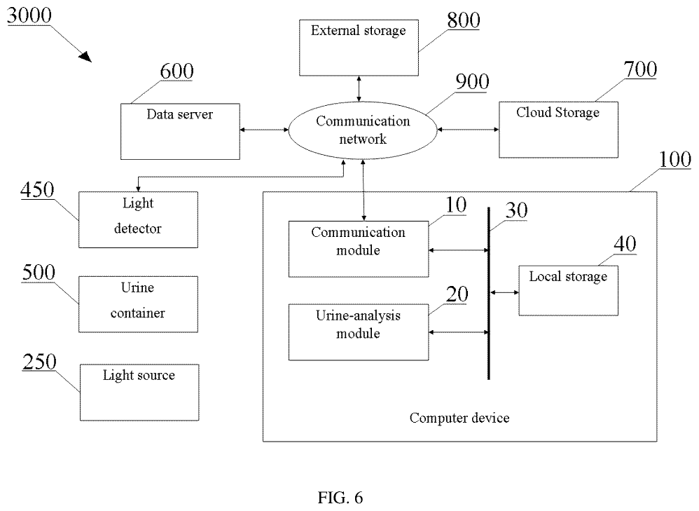

FIG. 6 is a block diagram illustrating a system 3000 for analyzing urine according to a seventh aspect of the present invention, wherein the urine-analyzing system 3000 is substantially an alternative variant of the urine-analyzing system 1000 shown in FIG. 1. Thus, the urine-analyzing system 3000 according to the seventh aspect of the present invention will be similar to the above-described urine-analyzing system 1000 according to the first aspect of the present invention, i.e. the urine-analyzing system 3000 according to the seventh aspect of the present invention has a structure, interconnections and main functional components similar to that of the urine-analyzing system 1000 according to the first aspect of the present invention (see FIGS. 1 and 5). In view of the above-mentioned similarity between the urine-analyzing system 3000 shown in FIG. 6 and the previously described urine-analyzing system 1000 shown in FIG. 1, most of details related to the urine-analyzing system 3000 according to the seventh aspect of the present invention are omitted in the present document and provided therein as a reference to corresponding description of the urine-analyzing system 1000 according to the first aspect of the present invention.

Thus, as compared to the urine-analyzing system 1000 provided with the above-described first and second light sources 200, 300, the urine-analyzing system 3000 shown in FIG. 6 comprises a light source 250. Generally speaking, the light source 250 comprised in the urine-analyzing system 3000 generally replaces the first and second light sources 200, 300 comprised in the urine-analyzing system 1000 and has a functionality similar to that of the light sources 200, 300.

In particular, in the urine-analyzing system 3000 shown in FIG. 6, the light source 250 is designed to emit a UV-VIS-NIR (Ultraviolet/Visible/Near-Infrared) light in a direction of the urine container 500, wherein the UV-VIS-NIR light has wavelength in the range of 200-1000 nm.

Furthermore, the as compared to the urine-analyzing system 1000 provided with the light detector 400, the urine-analyzing system 3000 shown in FIG. 6 comprises a light detector 450. Generally speaking, the light detector 450 comprised in the urine-analyzing system 3000 generally replaces the light detector 400 comprised in the urine-analyzing system 1000 and has a functionality similar to that of the light detector 400.

In particular, in the urine-analyzing system 3000 shown in FIG. 6, the light detector 450 is designed to detect the UV-VIS-NIR light transmitted or passed through urine in the urine container 500 to generate a single urine spectrum.

Similar to the urine-analyzing system 1000 shown in FIG. 1, the computer device 100 shown in FIG. 6 is connected to the light detector 450 to receive the generated urine spectrum therefrom. Also, similarly to the urine-analyzing system 1000 shown in FIG. 1, the computer device 100 shown in FIG. 6 is configured to receive personal patient data corresponding to the received urine spectrum from a data storage (e.g. the local storage 4, the data server 600, the cloud storage 700 or the external storage 800) and further configured to combine the received urine spectrum with the received personal patient data to generate a urine data matrix, wherein the computer device 100 shown in FIG. 6 is also further configured to feed the generated urine data matrix to one or more computing models stored in a local storage accessed by the computer device 100, each computing model being based on predetermined personal patient data and a predetermined correlation between a particular urine parameter obtained by a standard urine analysis method and measured urine spectra corresponding to the personal patient data, so as to determine at least one urine parameter. In other words, the urine parameters determined by the computer device 100 shown in FIG. 6 is a result or as an output provided by the computer device 100 shown in FIG. 6.

It is to note that the above-described particular or alternative embodiments of the urine-analyzing system 1000 are applicable to the urine-analyzing system 3000 shown in FIG. 6.

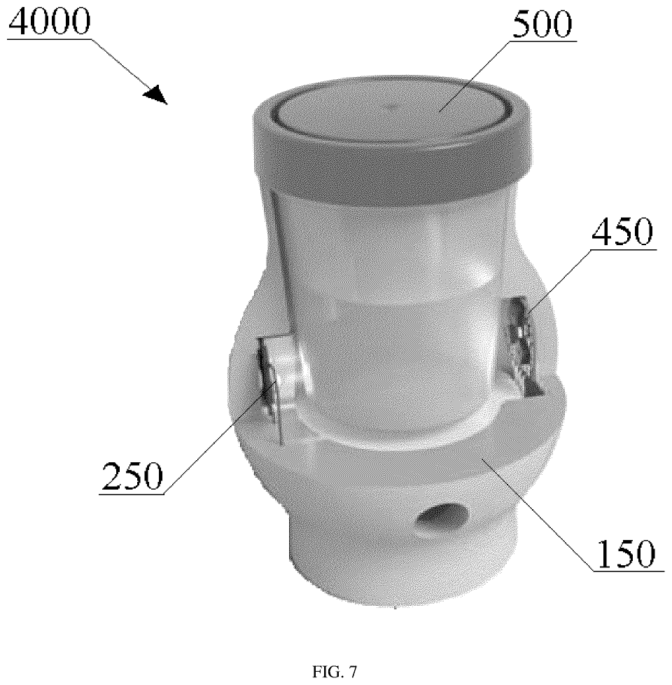

The first light 250 shown in FIG. 6, the light detector 450 shown in FIG. 6 and the computer device 100 shown in FIG. 6 as described above for the seventh aspect of the present invention may be corresponding functional components of an integral device 4000 for analyzing urine (also referred to in the present document as a urine-analyzing device 4000). FIG. 7 is a block diagram illustrating the urine-analyzing device 4000 according to the eight aspect of the present invention.

The urine-analyzing device 4000 shown in FIG. 7 is substantially an alternative variant of the urine-analyzing device 2000 shown in FIG. 2. Thus, the urine-analyzing device 4000 according to the eight aspect of the present invention will be similar to the above-described urine-analyzing device 2000 according to the second aspect of the present invention, i.e. the urine-analyzing device 4000 according to the eight aspect of the present invention has a structure, interconnections and main functional components similar to that of the urine-analyzing device 2000 according to the second aspect of the present invention (see FIGS. 2 and 5). In view of the above-mentioned similarity between the urine-analyzing device 4000 shown in FIG. 7 and the previously described urine-analyzing device 2000 shown in FIG. 2, most of details related to the urine-analyzing device 4000 according to the eight aspect of the present invention are omitted in the present document and provided therein as a reference to corresponding description of the urine-analyzing device 2000 according to the second aspect of the present invention.

Thus, as compared to the urine-analyzing device 2000 provided with the above-described first and second light sources 200, 300 shown in FIG. 2 and with the light detector 400 shown in FIG. 2, the urine-analyzing device 4000 shown in FIG. 7 comprises the light source 250 described above for the urine-analyzing device 4000 and the light detector 450 described above for the urine-analyzing device 4000. In particular, the light source 250 shown in FIG. 7 is installed or mounted on the base 150 such that the UV-VIS-NIR light emitted by the light source 250 is directed to the urine container 500, and the light detector 450 is installed or mounted on the base 150 such that the UV-VIS-NIR light transmitted or passed through urine or urine sample contained in the urine container 500 is received or detected by the light detector 450. Also, the computer device 100 is installed or mounted inside the base 150 and substantially operates or functions in the same manner as the computer device 100 described above for the urine-analyzing system 3000 shown in FIG. 6. It is to note that the urine spectrum generated by the light detector 450 comprises spectral data related to a transmission spectrum, scattering spectrum, fluorescence spectrum and/or a luminescence spectrum.

In a preferred embodiment of the present invention, the urine spectrum generated by the light detector 450 is a transmission spectrum or scattering spectrum.

Due to objective similarity between the urine-analyzing device 4000 shown in FIG. 7 and the urine-analyzing device 2000 shown in FIG. 2, it is clear for the skilled person that the above-described particular or alternative embodiments of the urine-analyzing device 2000 are applicable to the urine-analyzing device 4000. In particular, in an embodiment of the present invention according to the eight aspect, the light source 250 may be a single light source or a combination of light sources. Further, in one embodiment of the present invention according to the eight aspect, the light source 250 may be a combination of narrowband light sources, each emitting light in a predetermined part of the UV-VIS-NIR light wavelength region. Further, in another embodiment of the present invention according to the eight aspect, the light detector 450 may be a broadband light detector designed to detect light in the UV-VIS-NIR light wavelength region, wherein the light source 250 is required to be a combination of narrowband light sources, each emitting light in a predetermined part of the UV-VIS-NIR light wavelength region. Further, in still another embodiment of the present invention according to the eight aspect, the light detector 450 may be a combination of narrowband light detectors, each being designed to detect light in a predetermined part of the UV-VIS-NIR light wavelength region.

In an embodiment of the present invention according to the seventh aspect, a diffuser (not shown) in the urine-analyzing system 3000 shown in FIG. 6 or the urine-analyzing device 4000 shown in FIG. 7 the may be installed or mounted on the base 150 between the urine container 500 and the light detector 450 (i.e. positioned in front of a sensing region of the light detector 450), wherein the diffuser is designed to scatter or homogenize the light passed through urine in the urine container 500. Similarly to the urine-analyzing system 1000 or the urine-analyzing device 2000, the diffuser in the urine-analyzing system 3000 shown in FIG. 6 or the urine-analyzing device 4000 shown in FIG. 7 reduces orientation-induced spectral distortions caused by the urine container 500 having a cylindrical wall provided with non-planar entry and exit surfaces for the transmitted light.

Further, in the eight aspect of the present invention, the urine-analysis module 20 in the computer device 100 may be further configured to retrieve or receive personal patient data (e.g. a patient height, weight, gender, age and/or diagnosis) from the local storage 40 via the communication bus 30 and combine them with the urine spectrum received from the light detector 450 in order to generate the urine data matrix to be fed to the computing models used by the urine-analysis module 20 shown in FIG. 7, so that the generated urine data matrix may further contain the received personal patient data or further based on the personal patient data (i.e. in addition to the urine spectrum received from the light detector 450).

In an alternative embodiment of the present invention according to the seventh aspect or eight aspect, each of the computing models used by the computer device 100 and provided with the urine data matrix previously generated by the computer device 100 may be alternatively based on predetermined personal patient data and a predetermined correlation between a particular disease diagnosed by a medical specialist or a standard medical diagnostic technique and measured urine spectra corresponding to the personal patient data, so that the computer device 100 may allow at least one disease of the patient or a plurality of diseases of the patient to be determined (i.e. allows detection of each of the required patient diseases). In other words, the computer device 100 according to the present alternative embodiment of the present invention substantially generally performs the most functionalities mentioned above for the system 3000 shown in FIG. 6, i.e. the computer device 100 also receives the urine spectrum corresponding to the light source 250 from the light detector 450 and receives predetermined personal patient data corresponding to the received urine spectrum from a data storage accessed by the computer device 100 to combine the received urine spectrum with the received personal patient data for generating the urine data matrix, however the above alternative computing models based on predetermined personal patient data and a predetermined correlation between measured urine spectra corresponding to the personal patient data and a particular disease diagnosed by a medical specialist or a standard medical diagnostic technique are applied to the generated urine data matrix, and patient diseases (i.e. not the at least one urine parameters related to the patient) are alternatively determined or detected as a result or an output provided by the computer device 100. Thus, in the present alternative embodiment of the present invention according to the seventh or eighth aspect, each of the computing models allows only corresponding one of the patient diseases to be determined within the urine analysis. Meanwhile, each of the computing models used by the urine-analysis module 20 contained in the computer device 100 according to the present alternative embodiment of the present invention is preliminarily trained on predetermined personal patient data and a plurality of measured urine spectra corresponding to the personal patient data, wherein each urine spectrum of said measured urine spectra corresponds to a patient having a particular diagnosed disease, and wherein said measured urine spectra correspond to patients having said diagnosed disease. Therefore, in the computer device 100 according to the present alternative embodiment of the present invention, each of the computing models used by the urine-analysis module 20 is preliminarily trained to detect a particular one of the patient diseases to be detected based on a single urine spectrum related to the urine sample and corresponding to the light source 250 and on personal patient data corresponding to the urine spectrum. It is clear for a skilled person that the computer device 100 in present alternative embodiment of the present invention according to the seventh or eighth aspect may be used as the above-described functional component of the system 3000 shown in FIG. 6 or used as a processing device in the urine-analyzing device 4000 shown in FIG. 7. It is to note that the present alternative embodiment of the present invention according to the seventh or eighth aspect actually discloses a system for analyzing urine according to a ninth aspect of the present invention and a urine-analyzing device according to a tenth aspect of the present invention.

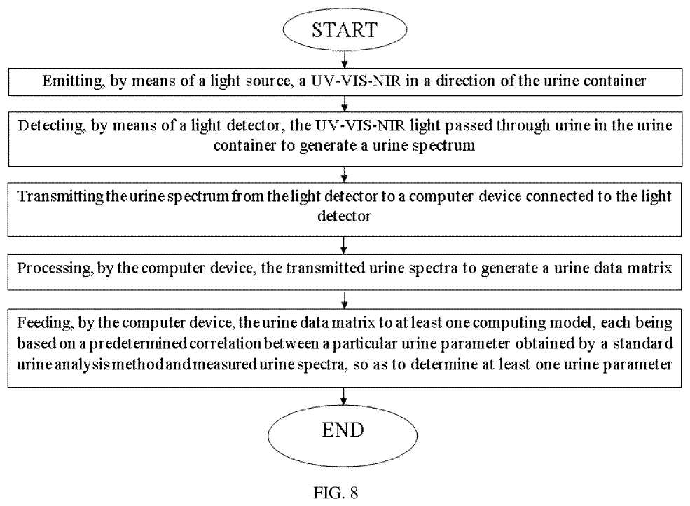

FIG. 8 illustrates a flow diagram of a method of analyzing a urine according to an eleventh aspect of the present invention.

The method of FIG. 8 may be implemented by the above system 3000 according to the seventh aspect of the present invention as shown in FIG. 6 or the above urine-analyzing device 4000 according to the eight aspect of the present invention as shown in FIG. 7. Anyway, the method of FIG. 8 may be implemented by any computing or electronic device known in the art, in particular by a processing unit of the above-mentioned general-purpose computer.

The method of FIG. 8 comprises the following stages or steps:
(1) emitting, by means of a light source, a UV-VIS-NIR light in a direction of the urine container;
(2) detecting, by means of a light detector, the UV-VIS-NIR light passed through urine in the urine container to generate a urine spectrum;
(3) transmitting the urine spectrum from the light detector to a computer device connected to the light detector;
(4) receiving, by the computer device, personal patient data corresponding to the transmitted urine spectrum from a data storage accessed by the computer device;
(5) combining, by the computer device, the transmitted urine spectrum with the received personal patient data to generate a urine data matrix; and
(6) feeding, by the computer device, the urine data matrix to one or more computing models stored in a local storage accessed by the computer device, each computing model being based on predetermined personal patient data and a predetermined correlation between a particular urine parameter obtained by a standard urine analysis method and measured urine spectra corresponding to the personal patient data, so as to determine at least one urine parameter.

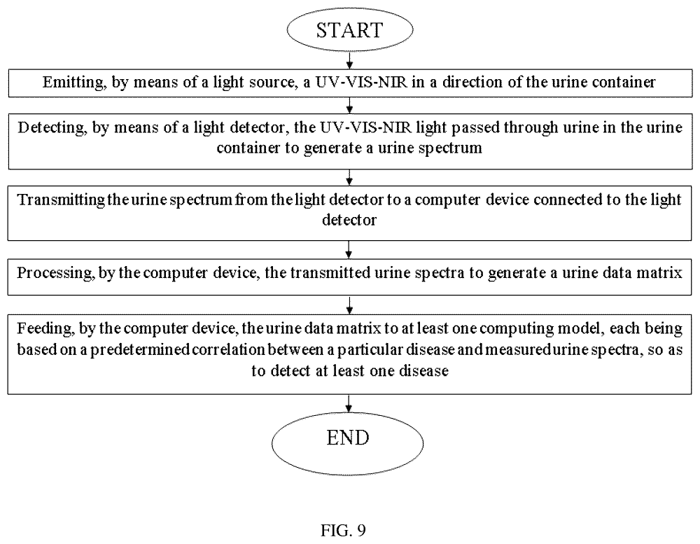

FIG. 9 illustrates a flow diagram of a method of analyzing a urine according to a twelfth aspect of the present invention.

The method of FIG. 9 may be implemented by the above urine-analyzing system according to the tenth aspect of the present invention or the above urine-analyzing device according to the eleventh aspect of the present invention. Anyway, the method of FIG. 9 may be also implemented by any computing or electronic device known in the art, in particular by a processing unit of the above-mentioned general-purpose computer.

The method of FIG. 9 comprises the following stages or steps:
(1) emitting, by means of a light source, a UV-VIS-NIR light in a direction of the urine container;
(2) detecting, by means of a light detector, the UV-VIS-NIR light passed through urine in the urine container to generate a urine spectrum;
(3) receiving, by the computer device, personal patient data corresponding to the transmitted urine spectrum from a data storage accessed by the computer device;
(4) combining, by the computer device, the transmitted urine spectrum with the personal patient data to generate a urine data matrix; and
(5) feeding, by the computer device, the urine data matrix to one or more computing models stored in a data storage accessed by the computer device, each computing model being based on predetermined personal patient data and a predetermined correlation between a particular disease and measured urine spectra corresponding to the personal patient data, so as to detect at least one disease.

It will be apparent to one of skill in the art that described herein is a novel system method and apparatus for free keystroke biometric authentication. While the invention has been described with reference to specific preferred embodiments, it is not limited to these embodiments. The invention may be modified or varied in many ways and such modifications and variations, as would be obvious to one of skill in the art, are within the scope and spirit of the invention and are included within the scope of the following claims.

We claim:

1. A device for analyzing urine, the device comprising:
a base (150) for receiving a urine container (500);
a first light source (200) designed to emit a NIR broadband light in a direction of the urine container (500);
a second light source (300) designed to emit a VIS broadband light in a direction of the urine container (500);
a light detector (400) configured to detect the NIR light passed through urine in the urine container (500) to generate a first urine spectrum and the VIS light passed through urine in the urine container (500) to generate a second urine spectrum, wherein
the first light source (200), the second light source (300) and the light detector (400) are mounted on the base (150) such that the received container (500) is positioned between the light sources (200, 300) and the light detector (400), and wherein the device further comprises
a processing device (100) connected to the light detector (400) to receive the first and second urine spectra therefrom, configured to receive personal patient data corresponding to the received urine spectra and configured to combine the received urine spectra with the received personal patient data to generate a urine data matrix,
wherein the processing device (100) is further configured to feed the urine data matrix to one or more computing models stored in a data storage accessed by the processing device (100), each computing model being based on predetermined personal patient data and a predetermined correlation between a particular urine parameter obtained by a standard urine analysis method and measured urine spectra corresponding to the personal patient data, so as to determine at least one urine parameter.

2. The device of claim 1, wherein the first and second light sources (200, 300) are both mounted on the same printed circuit board.

3. The device of any of claim 1, wherein each of the first and second urine spectra comprises spectral data related to a transmission spectrum, scattering spectrum, fluorescence spectrum and/or luminescence spectrum.

4. The device of claim 1, wherein the first light source (200) is a first combination of narrowband light sources, each emitting light in a predetermined part of a NIR wavelength region, and the second light source (300) is a second combination of narrowband light sources, each emitting light in a predetermined part of the VIS light wavelength region.

5. The device of claim 4, wherein the light detector (400) is single or multiple broadband light detectors designed to detect light in a NIR light wavelength region and detect light in a VIS light wavelength region.

6. The device of claim 1, wherein the light detector (400) is a combination of narrowband light detectors, each being designed to detect light in a predetermined part of the NIR light wavelength region or detect light in a predetermined part of the VIS light wavelength region.

7. The device of claim 1, wherein the personal patient data comprises height, weight, gender, age and/or diagnosis.

8. The device of claim 1, further comprising a diffuser positioned between the urine container (500) and the light detector (400), the diffuser being designed to scatter or homogenize the light passed through urine in the urine container (500), wherein the urine container (500) is cylindrically shaped, and the container (500) is received in the base (150) with a backlash.

9. The device of claim 1, wherein the processing device (100) is further configured to preliminary normalize each of the received first and second urine spectra by maximum intensity before generating the urine data matrix.

10. The device of claim 1, wherein the processing device (100) is configured to apply each of the computing models to the urine data matrix by categorizing the urine matrix according to pre-determined target urine parameters into a category of molecular-scale biomarkers and a category of cell-size biomarkers and by feeding the urine matrix to two PLS regression models, each corresponding to one of the determined urine parameter category and being based on a predetermined correlation between particular urine parameters obtained by standard urine analysis methods and measured urine spectra, for determining said at least one urine parameter.

11. The device of claim 1, wherein each of the computing models used by the processing device (100) for determining said at least one urine parameter is preliminary trained by using a training dataset formed of urine sample spectra obtained by a standard spectrometer and synthetic spectra, each synthetic spectrum being formed by combining at least two of the obtained urine sample spectra related to the same urine parameter category determined by the processing device (100).

12. A device for analyzing urine, the device comprising:
a base (150) for receiving a urine container (500);
a light source (250) designed to emit a UV-VIS-NIR light in a direction of the urine container (500);
a light detector (450) configured to detect the UV-VIS-NIR light passed through urine in the urine container (500) to generate a urine spectrum, wherein the light source (250) and the light detector (450) are mounted on the base (150) such that the received container (500) is positioned between the light source (250) and the light detector (450); and
a processing device (100) connected to the light detector (450) to receive the urine spectrum therefrom, configured to receive personal patient data corresponding to the received urine spectrum and configured to combine the received urine spectrum with the received personal patient data to generate a urine data matrix,
wherein the processing device (100) is further configured to feed the urine data matrix to one or more computing models stored in a data storage accessed by the processing device (100), each computing model being based on predetermined personal patient data and a predetermined correlation between a particular urine parameter obtained by a standard urine analysis method and measured urine spectra corresponding to the personal patient data, so as to determine at least one urine parameter.

13. The device of claim 12, wherein the light source is a single light source or a combination of light sources.

14. The device of claim 12, wherein the generated urine spectrum comprises data related to a transmission spectrum, scattering spectrum, fluorescence spectrum or luminescence spectrum.

15. The device of claim 12, wherein the light source (250) is a combination of narrowband light sources, each emitting light in a predetermined part of the UV-VIS-NIR light wavelength region.

16. The device of claim 15, wherein the light detector (450) is a broadband light detector designed to detect light in the UV-VIS-NIR light wavelength region.

17. The device of claim 12, wherein the light detector (450) is a combination of narrowband light detectors, each being designed to detect light in a predetermined part of the UV-VIS-NIR light wavelength region.

18. The device of claim 12, wherein the personal patient data comprises height, weight, gender, age and/or diagnosis.

19. The device of claim 12, further comprising a diffuser positioned between the urine container (500) and the light detector (450), the diffuser being designed to scatter or homogenize the light passed through urine in the urine container (500).

20. A method of analyzing a urine, the method comprising:
emitting, by a first light source (200), a NIR broadband light in a direction of a urine container (500);
emitting, by a second light source (300), a VIS broadband light in a direction of the urine container (500);
detecting, by a light detector (400), the NIR broadband light passed through urine in the urine container (500) to generate a first urine spectrum and the VIS light passed through urine in the urine container (500) to generate a second urine spectrum, wherein the first light source (200), the second light source (300) and the light detector (400) are mounted on the base (150) such that the received container (500) is positioned between the light sources (200, 300) and the light detector (400);
transmitting the first and second urine spectra from the light detector (400) to a computer device (100) connected to the light detector (400);
receiving, by the computer device (100), personal patient data corresponding to the received urine spectra from a data storage accessed by the computer device (100);
combining, by the computer device (100), the transmitted urine spectra with the received personal patient data to generate a urine data matrix; and
feeding, by the computer device (100), the urine data matrix to one or more computing models stored in a data storage accessed by the computer device (100), each computing model being based on predetermined personal patient data and a predetermined correlation between a particular urine parameter obtained by a standard urine analysis method and measured urine spectra corresponding to the personal patient data, so as to determine at least one urine parameter.

21. A method of analyzing a urine, the method comprising:
emitting, by a light source (250), a UV-VIS-NIR light in a direction of a urine container (500);
detecting, by a light detector (450), the UV-VIS-NIR light passed through urine in the urine container (500) to generate a urine spectrum, wherein the light source (250) and the light detector (450) are mounted on the base (150) such that the received container (500) is positioned between the light source (250) and the light detector;
transmitting the urine spectrum from the light detector (450) to a computer device (100) connected to the light detector (450);
receiving, by the computer device (100), personal patient data corresponding to the received urine spectrum from a data storage accessed by the computer device (100);
combining, by the computer device (100), the transmitted urine spectrum with the received personal patient data to generate a urine data matrix; and
feeding, by the computer device (100), the urine data matrix to one or more computing models stored in a data storage accessed by the computer device (100), each computing model being based on predetermined personal patient data and a predetermined correlation between a particular urine parameter obtained by a standard urine analysis method and measured urine spectra corresponding to the personal patient data, so as to determine at least one urine parameter.

22. A system for analyzing urine, the system comprising:
a first light source (200) designed to emit a NIR broadband light in a direction of a urine container (500);
a second light source (300) designed to emit a VIS broadband light in a direction of the urine container (500);
a light detector (400) designed to detect the NIR light passed through urine in the urine container (500) to generate a first urine spectrum and the VIS light passed through urine in the urine container (500) to generate a second urine spectrum, wherein the first light source (200), the second light source (300) and the light detector (400) are mounted such that the received container (500) is positioned between the light sources (200, 300) and the light detector (400); and
a computer device (100) connected to the light detector (400) to receive the first and second urine spectra therefrom, configured to receive personal patient data corresponding to the received urine spectra from a data storage accessed by the computer device (100) and configured to combine the received urine spectra with the received personal patient data to generate a urine data matrix, wherein
the computer device (100) is further configured to feed the generated urine data matrix to one or more computing models stored in a data storage accessed by the computer device (100), each computing model being based on predetermined personal patient data and a predetermined correlation between a particular urine parameter obtained by a standard urine analysis method and measured urine spectra corresponding to the personal patient data, so as to determine at least one urine parameter.

23. A system for analyzing urine, the system comprising:
a light source (250) designed to emit a UV-VIS-NIR light in a direction of a urine container (500);
a light detector (450) designed to detect the UV-VIS-NIR light passed through urine in the urine container (500) to generate a urine spectrum, wherein the light source (250) and the light detector (450) are mounted such that the received container (500) is positioned between the light source (250) and the light detector (450); and
a computer device (100) connected to the light detector (450) to receive the generated urine spectrum therefrom, configured to receive personal patient data corresponding to the received urine spectrum and configured to combine the received urine spectrum with the received personal patient data to generate a urine data matrix, wherein
the computer device (100) is further configured to feed the generated urine data matrix to one or more computing models stored in a data storage accessed by the computer device (100), each computing model being based on predetermined personal patient data and a predetermined correlation between a particular urine parameter obtained by a standard urine analysis method and measured urine spectra corresponding to the personal patient data, so as to determine at least one urine parameter.

24. A device for analyzing urine, the device comprising:
a base (150) for receiving a urine container (500);
a first light source (200) designed to emit a NIR broadband light in a direction of the urine container (500);
a second light source (300) designed to emit a VIS broadband light in a direction of the urine container (500);
a light detector (400) configured to detect the NIR light passed through urine in the urine container (500) to generate a first urine spectrum and the VIS light passed through urine in the urine container (500) to generate a second urine spectrum, wherein the first light source (200), the second light source (300) and the light detector (400) are mounted on the base (150) such that the received container (500) is positioned between the light sources (200, 300) and the light detector (400); and
a processing device (100) connected to the light detector (400) to receive the first and second urine spectra therefrom, configured to receive personal patient data corresponding to the received urine spectra and configured to combine the received urine spectra with the received personal patient data to generate a urine data matrix,
wherein the processing device (100) is further configured to feed the urine data matrix to one or more computing models stored in a data storage accessed by the processing device (100), each computing model being based on predetermined personal patient data and a predetermined correlation between a particular disease and measured urine spectra corresponding to the personal patient data, so as to detect at least one disease.

25. The device of claim 24, further comprising a diffuser positioned between the urine container (500) and the light detector (400), the diffuser being designed to scatter or homogenize the light passed through urine in the urine container (500).

26. A device for analyzing urine, the device comprising:
a base (150) for receiving a urine container (500);
a light source (250) designed to emit a UV-VIS-NIR light in a direction of the urine container (500);
a light detector (450) configured to detect the UV-VIS-NIR light passed through urine in the urine container (500) to generate a urine spectrum, wherein the light source (250) and the light detector (450) are mounted on the base (150) such that the received container (500) is positioned between the light source (250) and the light detector; and
a processing device (100) connected to the light detector (450) to receive the urine spectrum therefrom, configured to receive personal patient data corresponding to the received urine spectrum and configured to combine the received urine spectrum with the received personal patient data to generate a urine data matrix,
wherein the processing device (100) is further configured to feed the urine data matrix to one or more computing models stored in a data storage accessed by the processing device (100), each computing model being based on predetermined personal patient data and a predetermined correlation between a particular disease and measured urine spectra corresponding to the personal patient data, so as to detect at least one disease.

27. The device of claim 26, further comprising a diffuser positioned between the urine container (500) and the light detector (450), the diffuser being designed to scatter or homogenize the light passed through urine in the urine container (500).

28. A method of analyzing a urine, the method comprising:
emitting, by a first light source (200), a NIR broadband light in a direction of a urine container (500);
emitting, by a second light source (300), a VIS broadband light in a direction of the urine container (500);
detecting, by a light detector (400), the NIR broadband light passed through urine in the urine container (500) to generate a first urine spectrum and the VIS light passed through urine in the urine container (500) to generate a second urine spectrum, wherein the first light source (200), the second light source (300) and the light detector (400) are mounted on the base (150) such that the received container (500) is positioned between the light sources (200, 300) and the light detector (400);
transmitting the first and second urine spectra from the light detector (400) to a computer device (100) connected to the light detector (400);
receiving, by the computer device (100), personal patient data corresponding to the received urine spectra;
combining, by the computer device (100), the transmitted urine spectra with the received personal patient data to generate a urine data matrix; and
feeding, by the computer device (100), the urine data matrix to one or more computing models stored in a data storage accessed by the computer device (100), each computing model being based on predetermined personal patient data and a predetermined correlation between a particular disease and measured urine spectra corresponding to the personal patient data, so as to detect at least one disease.

29. A method of analyzing a urine, the method comprising:
emitting, by a light source (250), a UV-VIS-NIR light in a direction of a urine container (500);
detecting, by a light detector (450), the UV-VIS-NIR light passed through urine in the urine container (500) to generate a urine spectrum, wherein the light source (250) and the light detector (450) are mounted on the base (150) such that the received container (500) is positioned between the light source (250) and the light detector;
transmitting the urine spectrum from the light detector (450) to a computer device (100) connected to the light detector (450);
receiving, by the computer device (100), personal patient data corresponding to the received urine spectrum;
combining, by the computer device (100), the transmitted urine spectrum with the received personal patient data to generate a urine data matrix; and
feeding, by the computer device (100), the urine data matrix to one or more computing models stored in a data storage accessed by the computer device (100), each computing model being based on predetermined personal patient data and a predetermined correlation between a particular disease and measured urine spectra corresponding to the personal patient data, so as to detect at least one disease.

30. A system for analyzing urine, the system comprising:
a first light source (200) designed to emit a NIR broadband light in a direction of a urine container (500);
a second light source (300) designed to emit a VIS broadband light in a direction of the urine container (500);
a light detector (400) designed to detect the NIR light passed through urine in the urine container (500) to generate a first urine spectrum and the VIS light passed through urine in the urine container (500) to generate a second urine spectrum, wherein the first light source (200), the second light source (300) and the light detector (400) are mounted such that the received container (500) is positioned between the light sources (200, 300) and the light detector (400); and
a computer device (100) connected to the light detector (400) to receive the first and second urine spectra therefrom, configured to receive personal patient data corresponding to the received urine spectra and configured to combine the received urine spectra with the received personal patient data to generate a urine data matrix, wherein
the computer device (100) is further configured to feed the generated urine data matrix to one or more computing models stored in a data storage accessed by the computer device (100), each computing model being based on predetermined personal patient data and a predetermined correlation between a particular disease and measured urine spectra corresponding to the personal patient data, so as to detect at least one disease.

31. A system for analyzing urine, the system comprising:
a light source (250) designed to emit a UV-VIS-NIR light in a direction of a urine container (500);
a light detector (450) designed to detect the UV-VIS-NIR light passed through urine in the urine container (500) to generate a urine spectrum, wherein the light source (250) and the light detector (450) are mounted such that the received container (500) is positioned between the light source (250) and the light detector; and
a computer device (100) connected to the light detector (450) to receive the generated urine spectrum therefrom, configured to receive personal patient data corresponding to the received urine spectrum and configured to combine the received urine spectrum with the personal patient data to generate a urine data matrix, wherein
the computer device (100) is further configured to feed the generated urine data matrix to one or more computing models stored in a data storage accessed by the computer device (100), each computing model being based on predetermined personal patient data and a predetermined correlation between a particular disease and measured urine spectra corresponding to the personal patient data, so as to detect at least one disease.

32. The device of claim 2, wherein each of the first and second urine spectra comprises spectral data related to a transmission spectrum, scattering spectrum, fluorescence spectrum and/or luminescence spectrum.

33. The device of claim 4, wherein the light detector (400) is a combination of narrowband light detectors, each being designed to detect light in a predetermined part of the NIR light wavelength region or detect light in a predetermined part of the VIS light wavelength region.
